(19) United States
(12) Patent Application Publication
(10) Pub. No.: US 2005/0069528 A1
(43) Pub. Date: Mar. 31, 2005

(54) Mammalian endothelial cell model systems

(75) Inventor: Beverly Teicher, Ashland, MA (US)

Correspondence Address: BANNER & WITCOFF, 1001 G STREET N W, SUITE 1100, WASHINGTON, DC, 20001, US

(73) Assignee: Genzyme Corporation, Framingham, MA (US)

(21) Appl. No.: 10/922,154

(22) Filed: Aug. 20, 2004

Related U.S. Application Data
(60) Provisional application No. 60/496,676, filed on Aug. 21, 2003.

(51) Int. Cl. A61K 45/00; C12N 5/08

(52) U.S. Cl. 424/93.7; 435/372

(57) ABSTRACT

AC133⁺/CD34⁺ cells isolated from human bone marrow can be stimulated with the pro-angiogenic factors VEGF, bFGF, and heparin, resulting in the generation of a population of cells that is adherent and possesses many of the same properties as mature endothelial cell types, HMVECs and HUVECs. The newly-formed, endothelial-like cells are referred as adherent endothelial precursor cells (aEPCs); these cells appear to be intermediates between haematopoietic stem cells (HSCs) and mature endothelial cells. Direct comparison of aEPCs with HMVECs and HUVECs in several in vitro functional assays, such as tube formation, migration, invasion, and expression of cells surface markers, reveals differences and similarities. In a Matrigel™ matrix angiogenesis assay the aEPCs form vessels in vivo and interact with human ovarian cancer cells. Mouse cell lines that are useful models for tumor endothelial cells are identified by determining mRNA and protein expression levels of murine homologs of tumor endothelial markers. Mouse cell lines selected as models for tumor endothelial cells can be used to evaluate pro-angiogenic and anti-angiogenic factors.

A portion of the disclosure of this patent document contains material that is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

### FIELD OF THE INVENTION

The invention relates to model systems of tumor endothelial cells. More particularly it relates to model systems which can be used to evaluate potential anti-angiogenic and pro-angiogenic agents.

### BACKGROUND OF THE INVENTION

The importance of normal cells and tissues to support the growth of tumors has been recognized for centuries. The observations of Van der Kolk (1), Jones (2) and Paget (3) documented this knowledge in the clinical science literature. Algire and Chalkey (4) reported that host vascular reactions could be elicited by growing tumors and described in detail the extent and tumor-specific nature of the induction of host capillaries by transplanted tumors. The central hypothesis of Algire and Chalkey was that vascular induction by solid tumors may be the major distinguishing factor leading to tumor growth beyond normal tissue control levels. By the late 1960s, Folkman and his colleagues (5-7) had begun the search for a tumor angiogenesis factor (TAF) and in 1971 Folkman proposed “antiangiogenesis” as a means of holding tumors in a nonvascularized dormant state (8).

Investigators working in embryogenesis distinguished between angiogenesis, vessels arising from sprouts on existing vessels and vasculogenesis, vessels arising from endothelial progenitor cells (angioblasts) (Auerbach 1994, Luttun 2002). The abnormality of tumor vasculature and the value of working with fresh, non-cultured live endothelial cells isolated from solid tumors were recognized by cancer researchers (Modzelewski) and the role of endothelial precursor cells from bone marrow was recognized by researchers studying mammalian development (Lu 1996, Kaufman 2001). Asahara et al. (1997, 1999a&b) isolated putative endothelial precursor cells or angioblasts from human peripheral blood by magnetic bead selection and proposed a role for these cells in postnatal vasculogenesis and pathological neovascularization. It was later shown that recruitment of progenitor cells from the bone marrow requires the activity of matrix metalloproteinase-9 (MMP-9) mediation of the release of Kit-ligand (Heissig). Studies in allogeneic bone marrow transplant recipients confirmed that circulating endothelial precursor cells or angioblasts in peripheral blood originated from the bone marrow (Lin 2000). Gehling et al. (2000) identified CD34+/AC133+ progenitor cells from bone marrow as a subpopulation of cells that could differentiate in vitro into endothelial cells.

Recent studies have formally tied circulating endothelial precursor cells to the development of tumor vasculature (Peichev 2000, Rafii 2000, Gill 2001, Lyden 2001, De Bont 2001). Gill et al. (2001) found that in patients with vascular insult secondary to burns or coronary artery bypass grafting, there was an almost 50-fold increase in circulating endothelial precursor cells within the first 6 to 12 hours after injury and lasting 48 to 72 hours in parallel with the plasma levels of VEGF. A similar pattern of mobilization of endothelial precursor cells from the bone marrow was observed in mice after injection with VEGF. A similar pattern of mobilization of endothelial precursor cells from the bone marrow was observed in mice after injection with VEGF. Using the Id1^{+/-}Id3^{-/-} mutant mouse that has impaired tumor vascular growth, Lyden showed that transplantation with wild-type bone marrow or with VEGF-mobilized bone marrow stem cells allowed recruitment of endothelial precursor cells sufficient to support tumor growth (Lyden 2001). De Bont (De Bont 2001) found that when NOD/SCID mice bearing human Daudi non-Hodgkin's lymphoma xenografts were injected intravenously with human CD34+ hematopoietic stem cells or angioblasts that the injected human CD34+ cells homed to the tumor and differentiated along the endothelial lineage.

AC133⁺ multipotent human bone marrow progenitor cells exposed to VEGF in cell culture differentiate into CD34⁺/VE-cadherin⁺/VEGFR2⁺ cells or angioblasts (Reyes 2002). Upon maintenance in cell culture these cells will continue to differentiate toward a more mature endothelial phenotype. When human AC133⁺ progenitor cells were injected intravenously into NOD/SCID mice bearing subcutaneous murine Lewis lung carcinoma these cells contributed to the developing tumor vasculature. Further support for the notion that tumor vasculature arises, in part, through the process of vasculogenesis, comes from studies in which murine endothelial precursor cells from bone marrow, from peripheral blood and from tumor-infiltrating cells were isolated from mice bearing human breast carcinoma xenografts (Shirakawa 2002). The numbers of endothelial precursor cells were elevated in the tumor-bearing mice compared to normal mice. There were a significant number of endothelial precursor cells found in the human breast carcinoma xenografts and maturation and proliferation of these cells in the tumors was evident. Recently, it was reported that NOD/SCID mice transplanted with human bone marrow and bearing human Namalwa or Granta 519 Burkitt's lymphoma xenografts had a 7-fold increase in circulating endothelial precursor cells compared with non-tumor-bearing mice. Continuous infusion of endostatin into the tumor-bearing mice resulted in inhibition of the mobilization of endothelial precursor cell from the bone marrow.

To date, most research directed toward the development of antiangiogenic anticancer agents has utilized the cells readily at hand, primarily human umbilical vein endothelial cells (HUVEC) and human microvascular endothelial cells (HMVEC), as the cell-based models of the tumor endothelium (Auerbach 2000). It may be that human endothelial precursor cells are a more representative model of tumor endothelium.

The optimization of more relevant in vivo and in vitro models for studying angiogenesis and tumor progression is vital to the successful development of therapeutic compounds. While the ultimate focus is on human targets, most pre-clinical in vivo evaluation occurs in mice. Therefore, it is critical to acknowledge the importance of conservation between human and murine targets when evaluating compounds in vitro. Moreover, it is important to develop our understanding of murine systems and how they correspond to human systems.

The abnormality of tumor vasculature has been studied extensively in mouse models. Modzelewski et al. (10) isolated and characterized fresh tumor-derived endothelial cells from the syngeneic RIF-1 fibrosarcoma. The dynamics of blood vessel growth, structure, cellular composition and gene expression have been followed using the murine syngeneic mammary MCaIV adenocarcinoma, the murine syngeneic Lewis lung carcinoma and the murine transgenic RIP-Tag2 pancreatic islet cell tumor (Morikawa 2002, Thurston 1999, Brown 2001). The mobilization of bone marrow progenitor cells to circulation, the maturation of these cells into endothelial precursor cells and the incorporation of the endothelial precursor cells from circulation into tumor vasculature has been elucidated largely in mice (Lu 1996, Capillo 2003, Shirakawa 2002a, Luttun 2002, Shirikawa 2002b, Lyden 2001, Heissig 2002, Asahara 1999a, Asahara 1999b). These studies used murine syngeneic tumors, human tumor and bone marrow xenografts in immunodeficient mice and genetically engineered mice and have been used to recapitulate the dynamics of processes observed in human patients.

The field of cancer therapeutics has moved away from general proliferation related targets such as DNA and tubulin toward more non-traditional pathological processes including angiogenesis or immunomodulation, as well as more selective molecular targets. In most cases, efficacy in mouse models remains the critical determinant of whether a potential therapeutic moves into development in clinical trials. However, it has become evident that the homology between the murine and human proteins of specific molecular targets is, frequently, not sufficient to depend upon efficacy of agents selected for the murine target to translate into highly effective therapy in the human clinic. This is most evident in the selection of monoclonal antibodies where it is has become necessary to develop monoclonal antibodies to the murine homolog of the human target (Lyden 2001). Therefore, mouse and human agents need to be developed in parallel, so that the tumor-bearing mouse remains the critical efficacy hurtle. Likewise, it is important to define cell-based models of murine tumor endothelial cells that can be used to evaluate potential therapeutics in cell culture to select those most appropriate for in vivo testing.

The isolation and maintenance of a pure population of primary murine endothelial cells has proven to be difficult and has restricted the use of these cells in angiogenesis assay systems. To overcome these barriers, several groups have generated murine endothelial cell lines from cells isolated from the axillary lymph node (O'Connell 1990, 1991, 1993), embryonic yolk saks (Lu 1996), aorta, brain and heart capillaries (Bastaki 1997). In selecting the appropriate murine in vitro model of tumor endothelial cells, it is necessary to identify a murine endothelial cell line that maintains the expression of traditional endothelial cell markers as well as markers found to be expressed by human tumor endothelial cells (St. Croix 2000, Carson-Walter 2001).

O'Connell et al. 1990, 1991, 1993 (47-49) generated several murine endothelial cell lines from endothelial cells isolated from axillary and inguinal lymph nodes of adult C3H/HeJ mice and transformed the cells using simian virus 40. When implanted in mice, these immortalized endothelial cells produced nodules that displayed the characteristics of Kaposi's sarcoma, a multi-focal malignant neoplasm consisting of spindle cells and believed to be derived from endothelial cells. The SVEC4-10EE2 cell line was derived from the parent SVEC4-10 cell line by the extraction of tumors generated after the injection of the parent cell line in nude mice. These cells were then sub-cloned using conditioned medium from SVEC4-10EE2 to isolate a slower growing murine endothelial cell line designated SVEC4-10EHR1. IP2E4 and 3B11 cell lines were sub-cloned from ascites fluid generated in mice implanted with SVEC4-10EHR1 cells. The 2H11 cell line was sub-cloned from a nodular spindle-like tumor derived from the SVEC4-10EHR1 cell line. The 2F2B cell line was developed from a lobular cutaneous tumor resulting from the implantation of SVEC4-10EE2 cells. Each of the seven murine endothelial cell lines has distinctive cellular morphology as well as different endothelial cell characteristics, including the expression of von Willebrand factor (vWF/Factor VIII), the incorporation of acetylated low-density lipoprotein, the upregulation of class II major histocompatibility complex expression in response to interferon gamma, and the formation of tubular networks on Matrigel (O'Connell 1990, 1991, 1993).

There are several markers that have proven useful for identifying endothelial cells in situ and in vitro, including PECAM/CD31, vWF, P1H12 and VEGFR2 (21-24). These cell surface proteins have tissue-type and vasculature-specific expression patterns. Recently, several novel markers have been identified that are specific for human tumor endothelial cells (TEM) (St. Croix 2000). The TEM expression pattern for the murine homolog of several of these markers was determined in murine B16 melanoma and human HCT116 colon carcinoma xenografts by in situ hybridization (Carson-Walter 2001). There is a need in the art for means of identifying murine endothelial cell lines which are useful for studying the anti-angiogenic activity of therapeutic compounds in vitro.

### BRIEF SUMMARY OF THE INVENTION

In a first embodiment an isolated population of adherent human EPCs (aEPCs) is provided. The aEPCs are made by stimulating human bone marrow cells expressing endothelial lineage markers AC133 and CD34 with VEGF, bFGF, and heparin.

In a second embodiment of the invention an isolated population of adherent human EPCs (aEPCs) is provided. The aEPCs are capable of invading human ovarian cancer cells clusters in a three dimensional in vitro assay.

In a third embodiment of the invention a method is provided for evaluating test agents as pro-angiogenic or anti-angiogenic factors. An isolated population of adherent human EPCs is contacted with a test agent. Tubule formation, migration, or invasion by the isolated population contacted with the test agent is evaluated relative to an isolated population not contacted with a test agent. The test agent is identified as having potential use as a pro-angiogenic factor if it increases tubule formation, migration, or invasion; the test agent is identified as having potential use as an anti-angiogenic factor it decreases tubule formation, migration, or invasion.

In a fourth embodiment of the invention a method is provided for stimulating AC133⁺/CD34⁺ human bone marrow cells. The cells are cultured on a collagen-coated surface in the presence of FBS, VEGF, and heparin.

In a fifth embodiment of the invention a method is provided for stimulating AC133⁺/CD34⁺ human bone marrow cells. The cells are cultured on a fibronectin-coated surface in the presence of FBS, VEGF, FGF, and heparin.

In a sixth embodiment of the invention a model system for human vasculature is provided. A nude mouse is co-injected with reconstituted basement membrane matrix and an isolated population of adherent human EPCs.

In a seventh embodiment of the invention a model system for human vasculature is provided. The model system comprises a sample of Matrigel™ matrix and aEPCs which has been removed from a nude mouse.

In an eighth embodiment of the invention a method is provided for identifying a mouse cell line useful as a model for tumor endothelial cells. Expression of two or more murine tumor endothelial markers is determined in one or more mouse cell lines. A mouse cell line is selected which expresses at least two of the markers more than it expresses 18S RNA.

In a ninth embodiment a method of evaluating test agents as pro-angiogenic or anti-angiogenic factors is provided. A mouse cell line selected as a model for tumor endothelial cells is contacted with a test agent. Tubule formation, migration, or invasion by the mouse cell line contacted with the test agent is evaluated relative to the mouse cell line not contacted with a test agent. The test agent is identified as having potential use as a pro-angiogenic factor if it increases tubule formation, migration, or invasion; the test agent is identified as having potential use as an anti-angiogenic factor it decreases tubule formation, migration, or invasion.

### BRIEF DESCRIPTION OF THE DRAWINGS

FIG. 1 shows generation times for each of seven murine endothelial cell lines at three concentrations of fetal bovine serum (2, 5 and 10%) determined over a 96 hour period. Generation times (hours) were calculated by applying an exponential curve fit to the growth curve data and extrapolating cell number doubling time in the presence of each serum concentration. The data are the means and SD of three independent experiments.

FIG. 2 shows inverted, phase microscope images of each of seven murine endothelial cell lines (2×10⁴ cells/well) in a tube formation assay on a thick layer of Matrigel for 5 hours at 37° C.

FIG. 3 shows the relative mRNA expression of the murine homologs of recognized endothelial cell surface markers determined by real time-PCR. The results were normalized to 18S mRNA expression and compared to the expression of the same human endothelial cell surface markers by human microvascular endothelial cells (HMVEC). The data are the means for two independent experiments.

FIG. 4 shows the relative mRNA expression of the murine homologs of tumor endothelial cell surface markers determined by real time-PCR. The results were normalized to 18S mRNA expression and are expressed relative to the expression level of each marker in 2H11 cells. The data are the means for two independent experiments.

FIG. 5 shows in situ hybridization of subcutaneously grown syngeneic mouse tumors: B16 melanoma, Lewis lung carcinoma and CT-26 colon carcinoma. Tissue sections (5 mm) of each tumor were stained with hematoxylin and exposed to riboprobes for mouse VEGFR2, mTEM 1, mTEM 7, mTEM 8, mTEM 9 and mTEM 3 and visualized by amplification with fast red chromagen. Images were taken at 20× magnification.

FIG. 6 shows growth curves for EPC, HUVEC and HMVEC under optimal culture conditions over the course of 96 hrs. The left panel shows cellular proliferation beginning with 2,000 cells per well. The right panel shows cellular proliferation beginning with 3,000 cells per well. The data are the mean±SD for three independent experiments.

FIG. 7 shows a schematic showing overlaps in gene expression determined by SAGE analysis for tumor endothelial cells derived from human surgical specimens of 3 breast tumors, 3 brain tumors and 1 colon tumor, and EPC and HMVEC grown in cell culture. The SAGE gene expression data from the 7 tumor endothelial cell libraries were pooled and genes expressed at higher levels in the tumor endothelium compared with normal endothelial cells derived from 1 normal breast, 2 normal brain and 1 normal colon specimen at >99%, >95% and >90% confidence levels by Chi Square analysis.

FIG. 8 shows EPC, HUVEC and HMVEC evaluated for their ability to invade through a layer of Matrigel™. EPC derived from more than one donor showed similar results. Invasion by HMVEC is greater than the invasion by EPC from donor 2 at 24 hrs. with p<0.01 and at 48 hrs. with p<0.05. The data are the mean±SD for three independent experiments.

FIGS. 9A and 9B show: FIG. 9A.) Time course of EPC migration in response to various concentrations of fetal bovine serum. The data are the mean±SEM for three independent experiments. Migration toward a serum stimulus is significantly greater at 4 hrs. with p<0.001 and migration toward 5% FBS is significantly greater than migration in the absence of serum stimulus at 24 hrs with p<0.05. FIG. 9B.) Comparison between EPC, HUVEC and HMVEC in the migration assay. The effect of VEGF¹⁶⁵, rhbFGF and heparin on the migration of EPC was also investigated. The data are the mean±SD for three independent experiments.

FIG. 10 shows the ability of EPC, AC133+/CD34+ bone marrow progenitor cells, HUVEC and HMVEC to form tubes/networks on Matrigel™ over 24 hours were compared. The tubes were visualized using calcein staining and Scion image analysis to derived pixel area. The data are the mean±SD for three independent experiments.

FIG. 11 shows SKOV3 human ovarian cancer cells imbedded in a collagen plug surrounded by Matrigel upon which were plated EPC or HMVEC labeled with green fluorescent PKH67 in a 24 well plate (Walter-Yohrling). Fluorescence was used to locate the EPC or HMVEC in the cultures after 48 hours. Fluorescence inside SKOV-3 cancer cell clusters indicate invasion by EPC while that lack of fluorescence in the HMVEC containing wells indicate inability of the HMVEC to invade.

FIGS. 12A-12E shows EPC pre-labeled with DAPI mixed with Matrigel™ and injected subcutaneously into nude mice in a 500 ml volume to form a plug. FIG. 12A.) Image of a hematoxylin and eosin stained section of an EPC containing Matrigel™ plug at 20× magnification shows tube/network formation after 7 days in vivo. FIG. 12B.) Image of a hematoxylin and eosin stained section of an EPC containing Matrigel™ plug at 40× magnification. The pink web-like pattern is the remaining Matrigel. FIG. 12C.) Fluorescence image of DAPI-stained EPC in a Matrigel™ plug at 40× magnification. FIG. 12D.) Fluorescence image of human CD31 staining in red (Cy3) and DAPI staining of all nuclei in blue. FIG. 12E.) Fluorescence image of human von Willebrand factor (Factor VIII) staining in red (Cy3) and DAPI staining of all nuclei in blue.

### DETAILED DESCRIPTION OF THE INVENTION

It is a discovery of the present inventors that cell populations of both human and murine origin can be isolated or identified that have use as model systems for studying angiogenesis. These model systems can also be used to identify potential therapeutic agents which are either pro- or anti-angiogenic. Pro-angiogenic agents have use inter alia for wound healing, whereas anti-angiogenic agents have use inter alia for cancer treatment.

An isolated population for use in the present invention preferably has at least 10⁵ cells, preferably at least 10⁶, and more preferably at least 10⁷, 10⁸, or 10⁹ cells. The population is preferably homogeneous, i.e., the cells within the population share the same properties and express the same markers. As a starting material for obtaining the human cells of the present invention one can purchase or isolate human bone marrow cells which express haematopoieteic stem cell lineage markers AC133 and CD34. One commercial source for such cells is Cambrex, Inc. (East Rutherford, N.J.). Fluorescence activated cell sorting can be used to isolate cells bearing the appropriate markers. Bone marrow cells can be isolated from humans according to well known techniques for bone marrow biopsy.

Murine cells for screening can be obtained from commercial or other pre-existing sources, such as cell culture collections. Alternatively, murine cell lines can be made, for example by transforming mouse cells with transforming viruses or transforming viral products, such as T antigen.

Adherent cells are those that grow attached to a surface of the growth chamber. Typically such surfaces are coated with a substance to enhance such adherence. Suitable substances include fibronectin, collagen, and laminin. Repeated passage of the adherent cells is possible. The adherent cells can be passaged at least 1, 3, 5, 7, 9, or 11 times.

Characteristic angiogenic functions include tubule formation, migration, and invasion. These functions can be evaluated in vitro using assays which are known in the art. These assays can be used to monitor perturbation caused by exogenously added test agents. The test agents can either enhance or inhibit the angiogenic function. While particular assays are described in the examples for each of these angiogenic functions, modifications and variations can be made to the protocols without departing from the spirit of the invention.

Not only can the adherent EPCs of the present invention be useful for in vitro assays and evaluations, but they can also be used in in vivo systems. The cells can be injected into a nude mouse, for example, in a matrix of reconstituted basement membrane matrix. The mouse can then be treated with various test agents. The effect of the test agents on the injected adherent EPCs can be observed for example, by removing a plug (a solid or semi-solid core of gelled matrix) and examining the growth habit of the adherent EPCs within the plug. To facilitate observation of the adherent EPCs, they can be labeled with any convenient label. Preferably the label does not harm the cells and is readily detectable. One suitable label which can be used is 4′,6-Diamidino-2-phenylindole (DAPI), dimethylsulfoxide.

Mouse cell lines suitable for use as model systems for tumor endothelial cells can be identified by determining if the cell lines express orthologues of the markers identified in human tumor endothelial cells. The markers can be any of those which are expressed in tumor endothelial cells significantly more than in normal endothelium. Such markers include mTEM1, mTEM3, mTEM5, mTEM7, and mTEM8. The tumors can be from the colon or other tissue, including breast, brain, prostate, liver, stomach, ovary, uterus, cervix, etc. Mouse cell lines that are up-regulated for expression of at least 2, 4, 6, 8, or 10 of these orthologues can be used as model systems. One way to determine a base line for expression is to compare expression of the orthologue to expression of a gene which is expressed consistently throughout the cell cycle. Genes which are thought to be minimally regulated throughout the cell cycle should be checked under the conditions of the particular assay to be used.

Progenitor cells derived from adult human bone marrow or from umbilical cord blood can be recruited into circulation and give rise to well-differentiated cell types. VEGF and bFGF in particular have been implicated in the differentiation of these circulating progenitor cells into endothelial cells (29). VEGF has been shown to modulate postnatal EPC kinetics in normal mice by increasing migration and chemotaxis (15, 16). SDF-1 and other cytokines upregulate MMP-9 which is required in the recruitment of hematopoietic stem cells and endothelial precursor cells from bone marrow (17). IGF-1, G-CSF, and SCGF also can drive progenitor cell toward an endothelial phenotype. Thus, EPC maturation can occur under a multitude of conditions supporting the notion that the multi-faceted potential these progenitor cells possess enables them to function and respond to different pathological settings.

Endothelial cells can arise from a subset of common hematopoietic stem cell precursors identified by the markers AC133 and CD34. In cell culture upon exposure to VEGF¹⁶⁵, rhbFGF and heparin, the loss of AC133 expression occurs early as the progenitor cells differentiate through stages to a cell with a phenotype resembling endothelial cells, herein described as aEPC. The aEPC generated express several molecular markers associated with endothelial cells such as P1H12, VEGFR2, PECAM and endoglin and demonstrate migration properties very similar to HUVEC and HMVEC (30, 31, 39). However, other endothelial cell markers such as thrombomodulin, ICAM1, ICAM2 and VCAM1 were not found on aEPC. The adhesion molecules ICAM and VCAM mediate the interaction between endothelial cells and T-cells and NK cells as well as between endothelial cells and stromal tissue or cancer cells (40). Thrombomodulin is a cell surface anticoagulant that modulates the activity of the hemostatic protease thrombin and blocks thrombin's procoagulant effects and enhances thrombin-dependent activation of anticoagulant protein C (41, 42).

Endothelial progenitor cells derived from the bone marrow can be found in circulation in adults and may be recruited to and incorporated into neovascularization at sites of wounds and tumor vascularization (10, 15, 16). Endothelial progenitor cells isolated from the circulation have a relatively high proliferation rate compared with mature endothelial cells shed from the blood vessel wall (18). In the cancer patient populations, circulating endothelial progenitor cells and aEPC may be utilized as a surrogate marker/biomarker of response to an antiangiogenic or cytotoxic therapy. Endothelial progenitor cells and aEPC have been identified in circulation and in the vicinity of the malignant cells in patients with multiple myeloma, astrocytoma and inflammatory breast cancer and additional malignancies will no doubt be identified (47-49). Shirakawa et al. (47) found a significantly higher population of tumor-infiltrating endothelial cells or endothelial precursor cells in tumor-associated stroma of inflammatory breast cancer specimens than in non-inflammatory breast cancer specimens using immunohistochemcial staining for Tie2, VEGF, Flt-1 and CD31. There is potential value for aEPC outside the field of oncology for treating vascular diseases by engraftment or as drug delivery systems. Continued characterization of aEPC, effects of cytokines and growth factors on aEPC differentiation, and identification of the capabilities of aEPC in preclinical and clinical settings will continue is important.

Like other areas of drug discovery, the field of antineoplastic antiangiogenic drug discovery has been hampered by the use of non-ideal models for human tumor vasculature and endothelium. The cell-based models that have been the standard for the field, HUVEC and HMVEC, are mature, well-differentiated, normal human endothelial cells. Through the study of genes expressed in human tumor endothelial cells isolated from fresh surgical specimens of human tumors and corresponding normal tissues as determined by SAGE analysis, it is clear that human tumor endothelium is not well represented by HUVEC and HMVEC. The search for better cell-based models for human tumor endothelial cells has yielded the aEPC which in cell culture were developed from AC133+/CD34+ bone marrow progenitor cells. The aEPC retain the basic functions of migration and tube formation and have greater proliferative capacity and greater invasive capacity than HUVEC and HMVEC. Recently endostatin has been shown to inhibit the mobilization of murine EPC in tumor-bearing mice into circulation and to reduce the effectiveness of xenotransplantation of human bone marrow cells into SCID mice (27). Utilization of aEPC rather than HUVEC or HMVEC in drug discovery for evaluating potential antiangiogenic therapies in the preclinical setting may result in the selection of targets and agents that will prove to be more effective in the clinic.

The identification of a murine endothelial cell line that has tumor endothelial marker expression is important for the appropriate analysis of tumor angiogenesis and potential antiangiogenic anticancer strategies in vitro. Experiments have identified the 2H11 immortalized murine endothelial cells to be a relevant murine model for tumor endothelial cells because 2H11 cells express many of the murine homologs of standard endothelial cell markers, including CD34/sialomucin, CD36/GPIIIB, CD105/endogolin, CD146/P1H12, and CD106/VCAM1, as well as several murine homologs of tumor endothelial markers. Like normal endothelial cells, the 2H11 cells will form a cellular network when grown on extracellular matrices.

The variety of endothelial cell markers that were studied represent many aspects of endothelial cell function but not all are specific for endothelial cells. CD31/PECAM-1 is also expressed by platelets, monocytes, neutrophils and selected T-cell subsets. The CD31/PECAM-1 protein plays a major role in the cell-cell interactions of endothelial cells and is widely accepted as a pan-endothelial marker of all types of endothelial cells (21, 22). CD34/sialomucin also participates in cell-cell interactions by playing a role in adherens junction formation and is primarily expressed by the tumor neovasculature (23). CD36/GPIIIB expression has been identified on human dermal microvascular endothelial cells as well as other non-endothelial cell types, including platelets and monocytes (24). The GPIIIB glycoprotein binds to extracellular matrix proteins including thrombospondin and collagen (25, 26) and is believed to play a role in the vascular complications associated with malaria (27). CD105/endoglin is related to the transforming growth factor-b type III receptor and has been found to be expressed by endothelial cells (28). It has been shown that CD105/endoglin plays a role in normal vascular architecture and has been found to be elevated in tumor endothelial cells in some systems (29, 30). Using antibodies selective for endothelin receptor B, Shetty et al. (31) found this receptor on the surface of vascular endothelial and smooth muscle cells and defined its role in mediating vasoregulatory activity. CD146/P1H12 is involved in calcium-independent homotypic microvascular endothelial cell adhesion and has become a widely used marker for microvascular endothelial cells (19, 32, 33). Tie1 and Tie2 are tyrosine kinase receptors for angiopoietin 1 and 2 believed to be specifically expressed on endothelial cells. The angiopoietin/Tie1 and Tie2 pathways are involved in embryonic and tumor angiogenesis mediating endothelial cell motility and recruitment of peri-endothelial cells (34-37). The adhesion molecule, CD106/VCAM-1, has been used as a marker for endothelial cells. In some systems, levels of VCAM-1 correlate with vascular injury and tumor progression (33, 3840). The VEGF pathway including VEGF and the receptors, VEGFR1 (flt-1) and VEGFR2 (KDR/flk-1), is critical in embryonic angiogenesis, normal and tumor angiogenesis (41). The VEGF receptors are expressed on varied cells, including monocytes (VEGFR1), neuronal precursor cells (VEGFR1 and VEGFR2) and podocytes (VEGFR2), but are most highly expressed on resting and active endothelial cells (42-44).

St. Croix et al. (50) identified genes that were upregulated in endothelial cells isolated from a human colon carcinoma as compared to endothelial cells isolated from normal colon mucosa from the same patient. The genes expressed by the colon tumor endothelial cells differed significantly from the genes expressed by HMVEC and HUVEC, the cells traditionally used when studying angiogenesis in vitro. Therefore, the identification of cells or cell lines with a gene expression profile more relevant to malignant disease is critical. Carson-Walter et al. (51) identified several human tumor endothelial markers predicted to be associated with the cell surface and verified the differential expression pattern of the homologs, mTEM1, mTEM5 and mTEM8 in murine tumor endothelial cells. The immortalized murine endothelial cell line, 2H11, expresses relatively high levels of mTEM1, mTEM5, mTEM7 and mTEM8, suggesting that these cells may be a useful model of tumor endothelial cells for cell-based assays.

The mouse remains the model species of choice in cancer experimental therapeutics. It is critical to the selection of potential therapeutics to be aware of the similarities and differences between the human and murine molecular targets. Because of the inter-species differences in specific molecular targets, it is often necessary to development potential therapeutic agents directed toward the murine protein. Whether syngeneic murine tumors or human tumor xenograft models are used, the stromal compartment of the tumors is murine. Several strategies have been used to transplant functional human endothelial cells into immunodeficient SCID mice such as subcutaneous implantation of a Matrigel matrix, collagen/fibronectin matrices or polylactic acid sponges containing genetically altered HUVEC or HMVEC (45-49). Alternatively, the SCID mouse has been successfully engrafted with human stem cells (50). Raychaudhuri et al. (51) developed a model that involves the transplantation of human psoriasis plaques into SCID mice that maintain the hyperproliferative characteristics of the psoriasis as well as a functional human vasculature. These methods allow human angiogenesis in a murine host but are not models of angiogenesis associated with malignant disease.

Animal models including genetically engineered mice and xeno-transplanted mice are being developed to facilitate the assessment of therapies directed toward human angiogenesis targets in the mouse. However, the mouse remains important in early preclinical development of potential antiangiogenic therapies. Cell-based models, including endothelial cell proliferation, migration and tube formation, are well-established as the primary screen for potential antiangiogenic activity. Performing these assays in both human and murine endothelial cells that have characteristics of human tumor endothelial cells from patients is the ideal. The immortalized murine 2H11 endothelial cell line appears to be an appropriate murine endothelial cell model of tumor angiogenesis.

While the invention has been described with respect to specific examples including presently preferred modes of carrying out the invention, those skilled in the art will appreciate that there are numerous variations and permutations of the above described systems and techniques that fall within the spirit and scope of the invention as set forth in the appended claims.

### EXAMPLES

### Example 1

### Murine Cell Line Evaluation

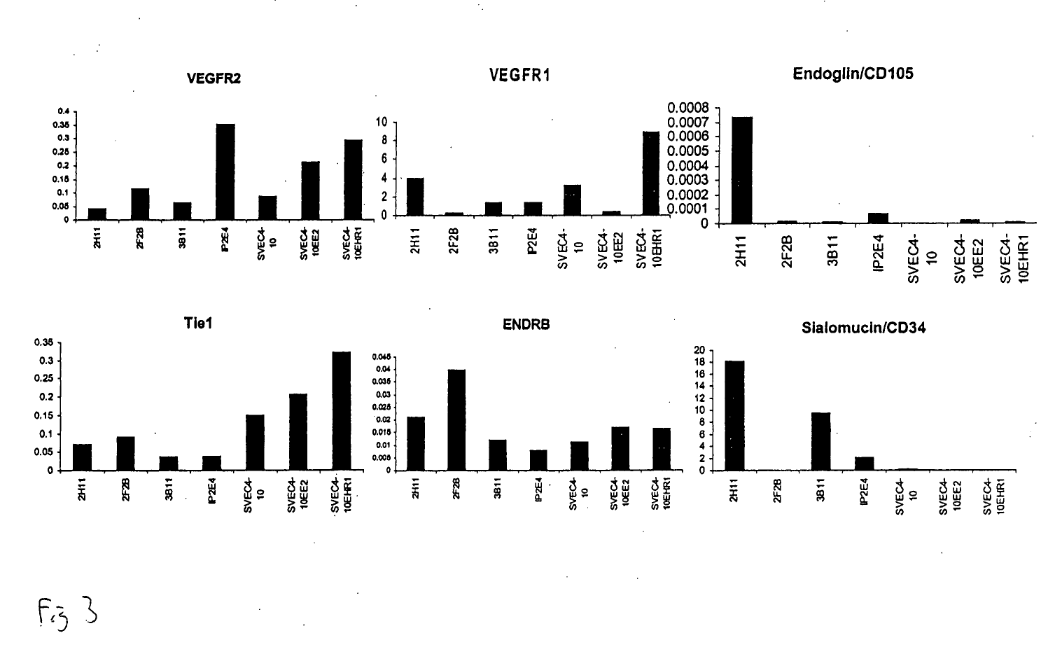

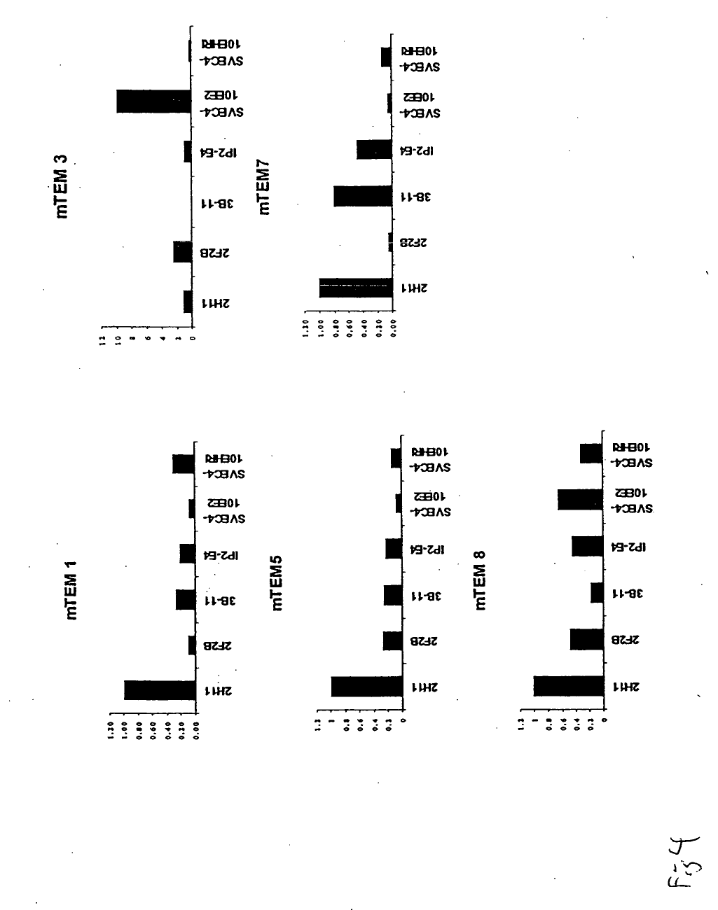

We evaluated the expression of known normal endothelial cell markers (Table 1), as well as tumor endothelial cell markers as defined by St. Croix, et al (Science, 289:1197, 2000) in several mouse endothelial cell lines. Real-time PCR and FACS analysis were used to evaluate mRNA (FIGS. 3 and 4) and protein expression of CD31, CD105, CD34, CD36, VCAM-1, CD141, ICAM1, ICAM2, EGFR, ENDRA, ENDRB, VEGFR2, VEGFR1, vWF, VE cadherin, Tie1, and Tie2 in these cells as compared to human dermal microvascular endothelial cells and human umbilical vein endothelial cells. In situ hybridization was also performed on tumor tissue from B16 and LLC syngeneic tumors to identify murine endothelial cell marker expression in vivo (FIG. 5).

Murine endothelial cell lines, SVEC4-10, SVEC4-10EE2, SVEC4-10EHR1, 2F2B, 2H11, IP1B, IP2-E4, were purchased from American Type Culture Collection (Manassas, Va.). Human dermal neonatal microvascular endothelial cells (HMVEC) and EGM2-MV were obtained from Cambrex (East Rutherford, N.J.). Cell culture media, versene and fetal bovine serum (FBS) were purchased from Invitrogen Corporation (Carlsbad, Calif.). Primary antibodies against endothelial cell markers were purchased from Pharmingen (San Diego, Calif.) and Chemicon (Temecula, Calif.) (Table 1). Phycoerythrin and fluoroscein-conjugated secondary antibodies were obtained from Jackson ImmunoResearch Laboratories (West Grove, Pa.). PCR primers that recognize the human and mouse gene were purchased from Integrated DNA Technologies (Coralville, Iowa). Trizol was purchased from Sigma (St. Louis, Mo.) and the RNA Extraction Kit was obtained from Qiagen (Valencia, Calif.). The High Capacity cDNA Archive Kit, Taqman Ribosomal RNA Control Reagents, Taqman Universal PCR Master Mix and Sybr Green PCR Master Mix were purchased from Applied Biosystems (Foster City, Calif.). Cell Titer Glo was purchased from Promega (Madison, Wis.).

The murine endothelial cell lines, 2F2B, 2H11, 3B11, IP2E4, SVEC4-10, SVEC4-10EE2 and SVEC4-10EHR1, were maintained in Dulbecco's modified Eagles medium (DMEM) plus 10% fetal bovine serum in a humidified 10% CO₂ environment. HMVEC were maintained in EGM2-MV that includes 5% FBS, VEGF, bFGF, EGF and IGF, in a humidified 5% CO₂ atmosphere.

TABLE 1
List of endothelial cell markers studied, the antibody used for FACS
analysis and primer sequences for mRNA expression analysis.

| EC Marker | Antibody | Forward Primer | Reverse Primer |
|---|---|---|---|
| CD34/sialoucin | Pharmingen cat | gaagacccttattacacgga | gctgaatggccgtttct |
| | # 553731 | (SEQ ID NO:1) | (SEQ ID NO:2) |
| CD36/GPIIIb | Chemicon cat | N/A | N/A |
| | # MAB1258 | | |
| CD105/endoglin | Pharmingen cat | cagcaagcgggagcccgtggt | ggtgctctgggtgctcccgat |
| | # 550546 | (SEQ ID NO:3) | (SEQ ID NO:4) |
| ENDRB | N/A | gcagaggactggccatttgga | gcaacagctcgatatctgtcaatact |
| | | (SEQ ID NO:5) | (SEQ ID NO:6) |
| P1H12/CD146 | Chemicon cat | N/A | N/A |
| | # MA816985 | | |
| Tie1 | N/A | tggagatagtgagccttgga | cagtttcgaggctgctccatgcg |
| | | (SEQ ID NO:7) | (SEQ ID NO:8) |
| Tie2 | Chemicon cat | gagattgttagcttaggaggcac | gtctcattagatcatacacctcatcat |
| | # MAB1148 | (SEQ ID NO:9) | (SEQ ID NO:10) |
| VCAM1/CD106 | Pharmingen cat | gacctgttccagcgagggtcta | cttccatcctcatagcaattaaggtgg |
| | # 553330 | (SEQ ID NO:11) | (SEQ ID NO:12) |
| VEGFR1 | N/A | ctgggagcctgcacgaagcaa | gtcacgtttgctcttgaggtagt |
| | | (SEQ ID NO:13) | (SEQ ID NO:14) |
| VEGFR2 | Pharmingen cat | gcagacagaaatacgtttgagttgg | agtgattgccccatgtgga |
| | # 555308 | (SEQ ID NO:15) | (SEQ ID NO:16) |

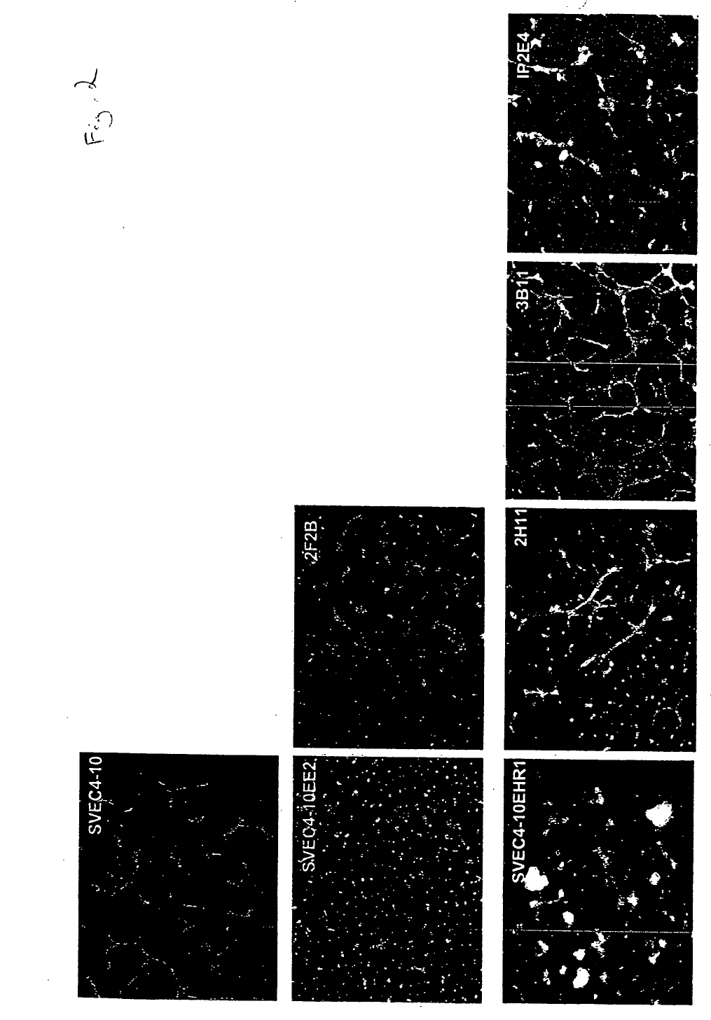

The assembly of cells into tubes/networks on a layer of extracellular matrix components is characteristic of endothelial cells in culture. The capability of each of the murine endothelial cell lines to assemble into tubes/networks was assessed in a tube formation assay on a layer of Matrigel over a five hour period (FIG. 2). Matrigel (BD Biosciences) was added to the wells of a 48-well plate in a volume of 120 μl and allowed to solidify at 37° C. for 30 minutes. After the Matrigel solidified, cells from each of the seven murine endothelial cell lines (2×10⁴ cells) were added in 200 μl of DMEM supplemented with 10% FBS. The cells were incubated at 37° C. with humidified 95% air/5% CO₂ for 5 hours.

The parental cell line SVEC4-10 was capable of tube formation. Neither one of the initial derivative lines, SVEC4-10EE2 or SVEC4-10EHR1, were able to form tubes on Matrigel. The second derivative line, 2F2B, that was derived from the SVEC4-10EE2 was able to form tubes on Matrigel, and each of the lines derived from the SVEC4-10EHR1 cells were also active in the tube formation assay.

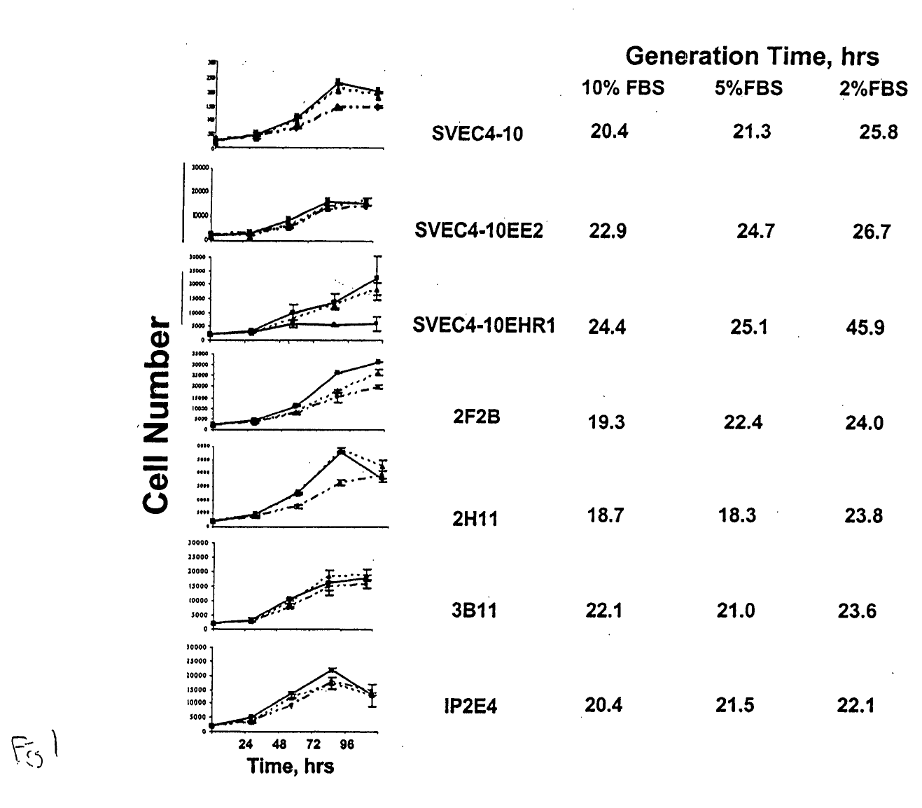

The generation times for each of the seven murine endothelial cell lines, 2H11, 2F2B, 3B11, IP2E4, SVEC4-10, SVEC4-10EE2 and SVEC4-10EHR1, were determined (FIG. 1). To determine cellular growth rates, cells from each of the seven murine endothelial cell lines (2×10³) were placed in each well of a 96-well plate in DMEM supplemented with 0, 2, 5 or 10% FBS. The cells were collected after 24, 48, 72 and 96 hours using Cell Titer Glo and extrapolating cell number from a standard curve for each cell line. Generation time was calculated by applying an exponential curve fit to the cell number values and calculating the time required for the cell number to double.

The effect of serum on the proliferation of each cell line was assessed at concentrations of 2, 5 and 10% FBS. Cell number was determined at 24, 48, 72 and 96 hours using standard curves derived from a metabolic luminescent endpoint. The effect of serum concentration on the cellular proliferation was small except with the slower growing SVEC4-10EHR1 cells whose generation time increased 1.8-fold when the cells were grown at the lowest serum concentration. In general the primary derivative cell lines, SVEC4-10EE2 and SVEC4-10EHR1, from the parental SVEC4-10 line were slower growing than the secondary derivative lines, 2F2B, 2H11, 3B11 and IP2E4. The cell line with the shortest doubling time was the 2H11 cells with a generation time of 18.7 hours at a serum concentration of 10% FBS and a generation time of 23.8 hours at a serum concentration of 2% FBS.

The mRNA expression of the murine homologs of recognized cell-surface endothelial cell markers in each of the seven murine endothelial cell lines was compared to the expression of the same marker in primary human microvascular endothelial cells (HMVEC) using primers that recognized both murine and human mRNAs (FIG. 3). The mRNA expression of each marker in the murine endothelial cell lines is represented as the expression relative to HMVECs. A confluent T75 flask of cells was harvested and lysed using Trizol. RNA was isolated by phenol chloroform extraction followed by column isolation using the Qiagen RNA Extraction Kit. cDNA was generated using the High Capacity cDNA Archive Kit. Real-time PCR for normal endothelial cell markers was performed with Sybr Green PCR Master Mix using the primers listed in Table 1 on an ABI Prism 7700 Sequence Detection System (Applied Biosystems, Foster City, Calif.). Relative mRNA expression of normal endothelial cell markers was determined by the delta-delta Ct method where the cycle threshold (Ct) of the sample is subtracted by the Ct of 18S and compared to the reference expression by HMVEC. Expression of the tumor endothelial cell markers was determined using FAM-labeled probes and Taqman Universal PCR Master Mix, expression was normalized to 18S and compared to the reference expression by 2H11 cells.

The expression of VEGFR2, VEGFR1 and Tie 1 by the murine endothelial cells was similar to the expression in HMVEC. The mRNA for endgolin/CD105 and ENDRB were found at much lower levels in the murine cell lines than in HMVEC and the mRNA for sialomucin/CD34 was found at much higher levels in the murine endothelial cells than in HMVEC. The SVEC4-10 parent cell line expressed VEGFR1, VEGFR2, ENDRB and Tie 1 but not endoglin/CD105 or sialomucin/CD34. The first derivative cell lines, SVEC4-10EE2 and SVEC4-10EHR1, had similar mRNA expression patterns to the parental cell line except that expression of VEGFR1 is markedly decreased and expression of endoglin/CD105 is increased in the SVEC4-10EE2 cells. The 2F2B cell line that was derived from the SVEC4-10EE2 cells also has decreased expression of VEGFR1 mRNA relative to the other murine endothelial cell lines. Only the 2H11 cell line and the two lines derived from ascites, 3B11 and IP2E4, expressed all six of the cell surface endothelial cell markers.

Since the goal is to identify a murine endothelial cell line that could be useful as a model for tumor endothelial cells, the mRNA expression of cell surface markers predicted to be selective for tumor endothelial cells was evaluated (FIG. 4). The expression of the mouse homologs of five tumor endothelial markers identified in endothelial cells isolated from human colon carcinoma were determined in six murine endothelial cell lines and values are relative to the expression in 2H11 cells. Overall, the 2H11 cell line was the highest expressor of these markers. The two lines derived from ascites, 3B11 and IP2E4, were also good expressors of all of the markers. The related lines SVEC4-10EE2 and 2F2B had variable expression of the markers and were particularly low expressors of mTEM7 and mTEM I. Based upon this analysis the murine 2H11 endothelial cell line appeared to be the most promising model for tumor endothelial cells.

Antibodies specific for the murine homologs for several recognized endothelial cell surface proteins are available. The cell surface expression of five endothelial cell markers was assessed for the seven murine endothelial cell lines and, using human specific antibodies, for HMVEC, by flow cytometry (Table 2). Cells were suspended by exposure to versene and 0.005% trypsin and then washed with PBS containing 5% FBS. Cells from each of the seven murine endothelial lines (2×10⁵) were incubated with 1 μg of primary antibody diluted in PBS containing 5% FBS in 100 μl total volume for 1 hour. The cells were washed twice in PBS containing 5% FBS and incubated with a 1:50 dilution of the appropriate fluorescently-conjugated secondary antibody for 1 hour. Samples were analyzed using a FACSCaliber flow cytometer (Becton Dickenson, Franklin Lakes, N.J.). The results were compared to appropriate isotype controls. Positive expression was determined by dividing the number of cells stained with the antibody by the total number of cells assayed multiplied by 100 (percent positive cells). Each determination was based on 10,000 events.

None of the seven murine endothelial cell lines expressed endoglin/CD105, while HMVEC were had strong expression of the endoglin protein. Three of the murine endothelial cell lines, 2H11, 3B11 and IP2E4, expressed sialomucin/CD34 protein as was reflected by the mRNA expression in these same three cell lines. All of the murine endothelial cells had some expression of gPIIIB/CD36. Only the 2H11 cell line expressed P1H12/CD146. Although all of the murine endothelial cell lines expressed VCAM1/CD106, the expression level for VCAM1 by the 2H11 and SVEC4-10EHR1 cell lines was most similar to that of the HMVEC

In situ hybridization was performed on tissue sections of three murine syngeneic tumors, B16 melanoma, Lewis lung carcinoma and CT-26 colon carcinoma grown subcutaneously in the appropriate hosts to determine the mRNA expression of the murine homologs of the known endothelial cell marker, VEGFR2, and the murine homologs of several tumor endothelial cell markers in the intratumoral vessels. Briefly, cDNA fragments were generated by PCR amplification of fragments ranging from 200 bp to 650 bp, using primers with T7 promoters incorporated into the antisense primers (19, 20). Digoxigenin riboprobes were generated by in vitro transcription in the presence of digoxigenin, according to manufacturer's instructions (Roche, Indianapolis, Ind.). Murine syngeneic tumors, B16 melanoma, Lewis lung carcinoma and CT-26 colon carcinoma, grown subcutaneously were harvested and prepared as formalin-fixed, paraffin embedded 3-5 mm sections on slides. All treatments were carried out at room temperature, unless otherwise stated. Sections were deparaffinized in xylene, washed in 100% ethanol, then hydrated in 85%, 75%, and 50% ethanol in distilled water. After incubation in DEPC treated water (Quality Biological, Inc., Gaitherburg, Md.), sections were permeabilized by treatment with pepsin in 0.2N hydrochloric acid, washed briefly in PBS then fixed in 4% paraformaldehyde. Sections were acetylated in acetic anhydride/0.1M triethanolamine, pH. 8.0, equilibrated for 10 minutes in 5×SSC (3 M sodium chloride, 0.3 M sodium citrate, pH 7.0; Invitrogen), and pre-hybridized for 1 to 2 hours at 55° C. in mRNA hybridization buffer (DAKO, Carpinteria, Calif.). Sections were hybridized with digoxigenin riboprobes (100-200 ng/ml) in mRNA hybridization buffer (DAKO) overnight at 55° C. After removing unbound riboprobes by washing, sections were incubated with RNase (Ambion, Austin, Tex.) to remove any non-specific bound riboprobe. Sections were treated with peroxidase block (DAKO) to eliminate any endogenous peroxidase, then blocked with a 1% blocking reagent (DIG nucleic acid detection kit, Roche), containing rabbit immunoglobulin fraction (DAKO), in Tris buffered saline. Rabbit anti-digoxigenin-HRP (DAKO) was used to detect the riboprobes, and served to catalyze the deposition of biotinylated tyramide (Gen-Point, DAKO) according to manufacturer's instructions. Additional amplification was accomplished through additional rounds of strept-av-HRP (GenPoint, DAKO) and biotinylated tyramide. Final detection was accomplished through rabbit anti-biotin conjugated to alkaline phosphatase (DAKO). Alkaline phosphatase was detected with Fast Red (DAKO) for 10-60 minutes at RT, then counterstained in hematoxylin. The nuclei were blued with ammonium hydroxide for 30 seconds, then mounted with crystal-mount (BioMeda, Foster City, Calif.). For the negative control sense riboprobes were used to detect any non-specific sequences. Additionally a slide that was exposed to RNAse to destroy the mRNA was hybridized with the anti-sense riboprobe to detect any non-specific hybridization.

The tumor endothelial cells were found to express VEGFR2 as well as mTEM1, mTEM3, mTEM5 and mTEM8 in all three in vivo mouse models of tumor vasculature as was previously shown by Carson-Walter et al. (20) (FIG. 5). Like Carson-Walter et al., we found that the B16 melanoma was negative for mTEM7, however, this marker was expressed by the vasculature of both the Lewis lung carcinoma and the CT26 colon carcnioma. The confirmation that the murine homologs of several markers identified in endothelial cells isolated from human tumors are expressed in these transplantable murine syngeneic tumors models validates their use in the study of tumor endothelial biology and provides guidance for selection of murine endothelial cell lines that would be appropriate models for tumor angiogenesis in cell culture.

The collected data suggest that of the cell lines we tested, 2H11 cells are the most relevant murine endothelial cells for studying tumor angiogenesis inhibitors in vitro. These cells can be used in in vitro angiogenesis assays (endothelial cell proliferation, invasion, migration, and tube formation assays) for evaluating potential pro- and anti-angiogenic properties and for evaluating inter-species activity of novel compounds.

### Example 2

### Cell Culture

CD34+/AC133+ progenitor cells from human bone marrow cells, human umbilical vein endothelial cells (HUVEC) and human microvascular endothelial cells (HMVEC) were purchased from Cambrex Inc. (East Rutherford, N.J.). The CD34+/AC133+ progenitors cells (1-2×10⁵ cells/ml) were grown in IMDM medium (Cambrex Inc.) supplemented with 15% fetal bovine serum (Invitrogen Corporation, Carlsbad, Calif.), 50 ng/ml VEGF₁₆₅ (R&D Systems, Minneapolis, Minn.), 50 ng/ml rhbFGF (R&D Systems), and 5 U/ml heparin (Sigma Chemical Co., St. Louis Mo.) on fibronectin coated flasks (BD Biosciences, Franklin Lakes, N.J.) at 37° C. with humidified 95% air/5% CO₂ to generate endothelial precursor cells (aEPC) (19, 29-31). Fresh media was added to the cultures every three to five days. The adherent cells that were generated from the original population of mixed non-adherent and adherent cells were designated aEPC. The aEPC were grown to confluency and could be passaged up to a dozen times. After the second passage, the aEPC were maintained in IMDM media supplemented with 15% FBS without additional growth factors. The aEPC were divided 2-3 fold at each passage. HUVEC and HMVEC were maintained in endothelial cell growth medium containing 2% FBS (EGM-2; Cambrex Inc.) at 37° C. with humidified 95% air/5% CO₂. Both of the donors for the AC133+/CD34+ progenitor bone marrow cells were normal male healthy volunteers. Both were Caucasian, ages 18 (donor 1) and 23 (donor 2) and both tested negative for HIV and hepatitis B and C infection.

The SKOV-3 human ovarian carcinoma cell line was purchased from American Type Culture Collection (Manassas, Va.). SKOV-3 cells were maintained in Dulbecco's Modified Eagle Medium (Invitrogen) supplemented with 10% FBS.

### Example 3

### Flow Cytometry

The expression of cell surface proteins that are characteristically expressed on bone marrow progenitor cells and mature endothelial cells was assessed on aEPC in the presence and absence of endothelial cell associated growth factors and on HUVEC and HMVEC (Table 3). aEPC, HMVEC, and HUVEC were collected by brief exposure to 0.25% tryspin/1 mM EDTA (Invitrogen Corporation) and washed twice in ice cold phosphate buffered 0.9% saline containing 5 mM EDTA and 5% FBS (FACs buffer). Approximately 2×10 5 cells were suspended in final volume of 100 μl of FACs buffer and incubated with a primary antibody for one hour on ice. The cells were then washed twice with FACs buffer and incubated with secondary antibody, when necessary, for 45 minutes on ice. The cells were again washed twice with FACs buffer and suspended in a final volume of 500 μl for flow cytometric analysis.

The following primary antibodies were used at a 1:20 dilution: 1.) anti-CD31-FITC (Pharmingen, San Diego, Calif.), 2.) anti-CD34-FITC (Pharmingen), 3.) anti-CD36-FITC (Pharmingen), 4.) anti-AC133-PE (Miltneyi Biotech, University Park, Pa.), 5.) anti-CD105 (Pharmingen), 6.) anti-P1H12 (Chemicon International, Temecula, Calif.), 7.) anti-CD54 (Pharmingen), and 8.) anti-CD106 (Pharmingen). The following un-conjugated primary antibodies were used at a 1:10 dilution: 1.) anti-VEGFR2 (Santa Cruz Biotechnology, Santa Cruz, Calif.), and 2.) anti-VE-cadherin (Santa Cruz Biotechnology). Unconjugated primary antibody against CD36 (Pharmingen) was used at a 1:100 dilution and antibody against CD141 (Pharmingen) was used at a 1:500 dilution. The following secondary antibodies were used at a 1:35 dilution: 1.) anti-mouse-FITC (Pharmingen or Jackson Immunoresearch, Bar Harbor, Me.), 2.) anti-rabbit-FITC (Santa Cruz) and 3.) anti-goat-FITC (Santa Cruz), or at a 1:50 dilution: 4.) anti-mouse-PE (Jackson Immunoresearch). aEPC and HUVEC were stimulated with 20 ng/ml TNF alpha (R&D Systems) for 48 hours prior to CD106 staining. Cells were fixed in 4% paraformaldehyde and analyzed within 24 hours. Positive expression was determined if cells gated at 10% or greater.

Flow cytometry was used to score relative expression of each marker on the various cell types. AC133/CD133 is a 97 kDa five-span transmembrane protein with no known function (37, 38). The expression of the AC133 protein is, in large part, limited to normal bone marrow and some CD34+ leukemias. The expression of progenitor cell marker AC133 on the cell-surface aEPC was weak. However, AC133 was not detectable on the surface of the mature endothelial cells represented by HUVEC and HMVEC. Sialomucin/CD34 was also a marker in the bone marrow progenitor cell population selected to be the originating cell for aEPC development. CD34 is found expressed in vessels in vivo and on about 20% of HUVEC and HMVEC in cell culture (39). aEPC, HUVEC and HMVEC in the current study were weakly positive for CD34 expression.

Several cell-surface endothelial cell markers were similarly expressed on the aEPC, HUVEC and HMVEC. Among the similarly expressed markers were VEGFR2/Flk-1, endoglin/CD105, and P1H12/CD146. VE-cadherin and gPIIIB/CD36 were expressed weakly in aEPC, HUVEC and HMVEC. Several markers differentiated aEPC from mature endothelial cells represented by HUVEC and HMVEC. ICAM1/CD54, ICAM2/CD102 and thrombomodulin/CD141 were much more strongly expressed on the mature endothelial cells than on the aEPC. Finally, PCAM/CD31 was very strongly expressed on the mature endothelial cells represented by HUVEC and HMVEC and was more weakly expressed on the surface of aEPC.

TABLE 2
Flow cytometry detection of murine homologs recognized endothelial cell
surface proteins. Murine endothelial cell lines were assessed for the
expression of endothelial cell marker proteins using antibodies directed
toward the mouse proteins. The same endothelial cells markers were
assessed on HMVEC using antibodies directed toward the human protein.
The data are presented as the percentage of the cell population
expressing the protein.

| | Endothelial Cell Surface Marker | | | | |
|---|---|---|---|---|---|
| | Sialomucin | GPIIIB | Endoglin | P1H12 | VCAM-1 |
| Cell Type | CD34 | CD36 | CD105 | CD146 | CD106 |
| 2H11 | 31 | 31 | 5 | 18 | 20 |
| 2F2B | 2 | 7 | 2 | 3 | 92 |
| 3B11 | 61 | 24 | 2 | 7 | 86 |
| IP2E4 | 12 | 28 | 4 | 7 | 86 |
| SVEC4-10 | 4 | 22 | 2 | 5 | 97 |
| SVEC4-10EE2 | 2 | 19 | 2 | 5 | 99 |
| SVEC4- | 2 | 24 | 2 | 9 | 36 |
| 10EHR1 | | | | | |
| HMVEC | 0 | 3 | 97 | 96 | 28 |

### Example 4

### In Vitro Tube Formation

HMVECs and HUVECs were maintained in EGM-2 media (Cambrex). Reconstituted basement membrane Matrigel™ matrix (BD Biosciences) was added to the wells of a 48-well plate in a volume of 150 uls and allowed to solidify at 37° C. for 30 minutes. After the Matrigel™ solidified, 15,000-20,000 cells were added in 300 uls of media: EGM-2 for HMVECs and HUVECs; IMDM with 2% FBS for aEPCs and bone marrow cells. Cells were incubated at 37° C. with 5% CO₂ and tube formation was imaged at 24 hours.

### Example 5

### Migration Assay and Invasion Assay

aEPC, HUVEC or HMVEC (5×10⁴ cells) were placed into the upper chamber of a BD Falcon HTS FluoroBlok insert with a PET membrane with eight micron pores (BD Biosciences) in 300 μl of serum free IMDM for aEPC, EGM-2 for HUVEC or HMVEC in triplicate. For the invasion assay the FluoroBlok inserts were coated with a thin layer of Matrigel. The inserts were placed into the lower chamber wells of a 24 well plate containing IMDM or EGM-2 media and FBS (0, 0.5 or 5%) as chemoattractant. For direct comparison of cell lines, five percent FBS was utilized. At 4, 24 and 48 hours, cells that migrated through the pores of the membrane to the lower chamber were stained with calcein 8 μg/ml (Molecular Probes, Eugene, Oreg.) in PBS for 30 minutes at 37° C. The fluorescence of migrated cells was quantified using a fluorimeter set at 485 nm excitation and 530 nm emission. Data are expressed as number of cells that migrated through or invaded pores +/−SD.

### Example 6

### Invasion Assay in Presence of Cancer Cell Clusters

Briefly, a thick layer of Matrigel (BD Biosciences) was added to the wells of a 24 well plate in a volume of 300 μl and allowed to solidify at 37° C. for 30 minutes. A plug of Matrigel of approximately 1 mm diameter was removed using a glass pipette under light vacuum. The hole was filled with SKOV3 cells suspended in 1% collagen I (Cohesion Technologies, Palo Alto, Calif.) at a concentration of 1×10⁶ cells in 5 μl and allowed to solidify at 37° C. for 30 minutes. aEPC or HMVEC were labeled with PKH67 green dye according to the manufacturer's instructions (Sigma). The cells were incubated in the presence of 2.5 μM dye suspended in diluent for 5 minutes. The labeling was stopped with 1 ml of FBS for 1 minute followed by 3 washes in serum-containing medium. Following the washes, the cells were suspended in IMDM or EGM-2 and counted by hemacytometer. aEPC or HMVEC (3×10⁵ cells) were added to each well in 300 μl of IMDM or EGM-2 media. The cultures were incubated at 37° C. in humidified 95% air/5% CO₂ for 24 or 48 hours. The aEPC or HMVEC in the wells were visualized using a fluoroscein (PKH67) filter on an inverted phase using a fluorescent inverted phase microscope.

### Example 7

### In Vivo Matrigel™ Matrix Angiogenesis Assay

aEPC were pre-labeled with DAPI (Sigma) at 20 μg/ml at 37° C. for 20 minutes, then were washed twice with PBS and used within 24 hours. The aEPC were collected by exposure to 0.25% trypsin. Approximately 5×10⁵ aEPC in 100 μl PBS were mixed with 500 μl of Matrigel (BD Biosciences) containing 40 U/ml heparin and 150 ng/ml rhbFGF. The Matrigel containing aEPC mixtures (500 μl) were implanted subcutaneously into the mid-dorsal region of female nude mice. The Matrigel plugs containing aEPC were collected after 7 days in vivo and snap frozen in OCT medium. For detection of DAPI-labeled aEPC, 5 μm frozen sections of the Matrigel plugs were rinsed briefly with PBS, fixed in 10% formalin for 10 minutes, washed twice, and then imaged by fluorescent microscopy after mounting. Other sections were stained with hematoxylin and eosin and imaged by bright field microscopy or stained with mouse anti-human CD31 (1 μg/200 μl/slide; clone JC70A; DAKO, Carpinteria, Calif.) or rabbit anti-human von Willebrand factor (DAKO) using a Cy3 secondary antibody for immunohistochemistry.

### Example 8

### Making Endothelial Precursor Cells

In the absence of stimulation, AC133+/CD34+ bone marrow cells can be maintained for a short period of time in culture with little expansion potential. Upon exposure to the angiogenic factors VEGF₁₆₅, rhbFGF and heparin, the AC133+/CD34+ progenitor cells began to proliferate. Within one to two weeks a new phenotype of cells began to emerge and adhere to the flask. After two weeks a confluent, adherent monolayer of elongated cells was obtained. These adherent cells derived by angiogenic growth factor exposure of AC133+/CD34+ progenitor cells in culture were designated endothelial precursor cells (aEPC). The remaining bone marrow cells in suspension that continued to proliferate and thrive were transferred to a new flask for additional exposure with the angiogenic factors VEGF¹⁶⁵, rhbFGF and heparin and generation of aEPC. The aEPC maintain significant expansion potential and can be passaged at least up to 12 times. For the experiments described herein, the aEPC were limited to 10 passages. Maintenance of the aEPC at a minimum of 50-60% confluence was important to generating high cell numbers with a doubling time of approximately 3 days. aEPC viability remained highest when at near confluence in culture.

The generation times of the aEPC, HUVEC and HMVEC were determined over a four day period. Cells were plated in 96 well plate format at 2,000 or 3,000 cells per well in triplicate. aEPC were grown in EGM-2 with 2% FBS or IMDM with 15% FBS and HMVEC and HUVEC were grown in EGM-2 media with 2% FBS. Cells were assayed daily using CellTiter-Glo Luminescent Cell Viability Assay (Promega, Madison, Wis.) that measures ATP levels. The results are expressed as cells per well +/−SD.

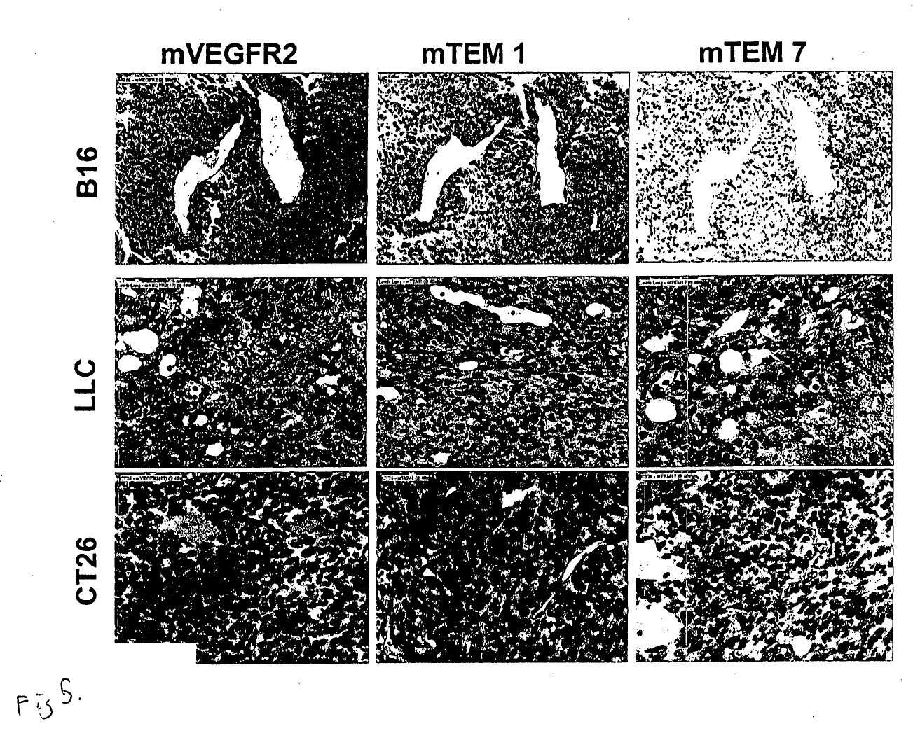

The performance of aEPC in several cell-based assays in comparison to the mature endothelial cells commonly utilized in the angiogenesis field, HUVEC and HMVEC was assessed. aEPC were evaluated in proliferation, migration, invasion through Matrigel and tube/network formation assays. These assays are routinely employed to identify and evaluate both pro-angiogenic and anti-angiogenic agents that may be potentially effective in therapeutic clinical settings. Generation times were determined for HMVEC, HUVEC, and aEPC over 96 hours (FIG. 6). aEPC were grown in either IMDM plus 15% FBS or in complete EGM-2 media that is supplemented with VEGF, bFGF and 5% FBS. The generation times for aEPC in IMDM media with high serum was approximately 117 hours, similar to HMVEC with a generation time of 115 hours. When aEPC are grown in EGM-2 media, the generation time decreased to approximately 36 hours, a rate more similar to HUVEC with a doubling time of approximately 27 hours. These results indicate that like mature endothelial cells, aEPC respond to growth factors such as VEGF and bFGF. aEPC proliferation rates resemble both HMVEC and HUVEC and comparisons are dependent upon the media chosen.

### Example 9

### Endothelial Cell Markers

The adherent EPCs were analyzed for the expression of cell surface antigens that are characteristically expressed on HMVECs and HUVECs by FACs analysis; data are shown in Table 3. Several of the markers were strongly expressed on all three cell types: CD31, CD105, VEGFR2, and P1H12. VEGFR2 and VE-cadherin were detected albeit at a lower level. The original stem cells markers AC133 and CD34 were undetectable on the adherent EPCs. Because AC133 is not detected in HMVECs or HUVECs, it is not surprising that its expression was down-regulated in the adherent EPCs. However, CD34 is commonly detected on adult endothelial cells in vivo. The failure to detect CD34 may be due to our culture conditions. Under the conditions that we used CD34 was also not detected in HMVECs or HUVECs.
50%, 3=51-75%, 4=76-100%. aEPC grown in the presence or absence of growth factors (GF; VEGF¹⁶⁵, rhbFGF and heparin) were compared.

Molecular Marker

| | AC133 | Sialoumucin | P1H12 | VEGFR2 | PCAM | Endoglin | VE- | ICAM1 | VCAM1 | ICAM2 | gPIIIB | thrombomodulin |
|---|---|---|---|---|---|---|---|---|---|---|---|---|
| Cell Type | CD133 | CD34 | CD146 | Flk1 | CD31 | CD105 | cadherin | CD54 | CD106 | CD102 | CD36 | CD141 |
| aEPC + | 1 | 1 | 3 | 3 | 2 | 4 | 1 | 1 | 0 | 1 | 1 | 1 |
| GF | | | | | | | | | | | | |
| aEPC | 1 | 1 | 4 | 3 | 3 | 4 | 1 | 1 | 2 | 1 | 1 | 1 |
| HUVEC | 0 | 1 | 4 | 3 | 4 | 4 | 1 | 3-4 | 4 | 4 | 1 | 4 |
| HMVEC | 0 | 1 | 4 | 3 | 3 | 4 | 1 | 2-3 | 0 | 4 | 1 | 4 |

aEPCs that had been utilized for FACs analysis had been maintained under standard conditions (media plus serum) or under stimulatory conditions (media plus serum with VEGF, FGF, heparin); little difference was observed between the two. The expression of von Willebrand factor was determined by RT-PCR; negligible amounts were detected. CD36 and VE-cadherin were analyzed by FACs but detected at minimal levels in all three cells lines. CD54 or ICAM was expressed in HUVECs but not in HMVECs or the aEPCs. CD106 or VCAM was detected in bone marrow-derived aEPCs following stimulation with TNF-alpha although expression was at lower levels compared to HUVECs.

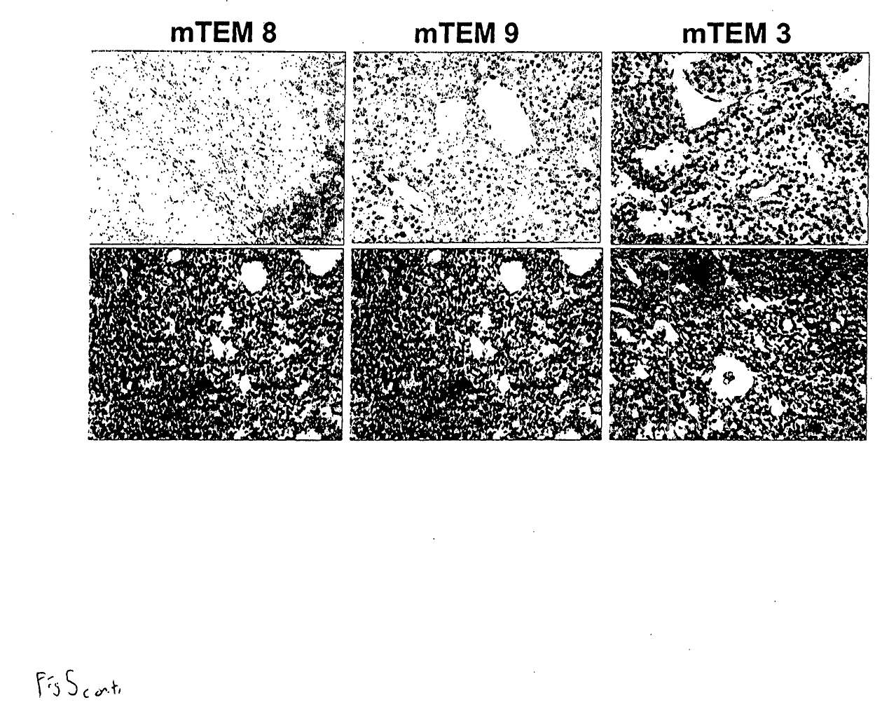

Gene expression in the aEPC was compared with gene expression from human tumor endothelial cells and with gene expression in HMVEC using SAGE analysis (33-34) (FIG. 7). SAGE libraries for brain, breast and colon tumor and normal endothelial cells (EC) and SAGE libraries from aEPC and HMVEC were constructed as previously described (32). SAGE libraries for brain, breast EC and aEPC were constructed using the long SAGE technology (33). SAGE libraries for colon EC and HMVEC were constructed using microSAGE technology (34). The sample information for all the libraries constructed is: 3 brain tumors and 2 normal brain samples, 2 primary breast tumors, 1 breast bone metastasis and 1 normal breast sample, 1 colon tumor and 1 normal colon sample, aEPC grown with or without VEGF, and HMVEC in the presence or absence of DMSO. SAGE tags were normalized to 50,000 total library counts except the colon EC libraries which were normalized to 100,000 total library counts. Long SAGE tags were converted to regular SAGE tags and tag counts for the same regular SAGE tags were aggregated. There were 139,838 unique SAGE tags from the 15 libraries. SAGE tag counts of 2 or less were filtered out in at least 10 of the 15 libraries to remove erroneous tags. Within tissue comparison of tumor vs. normal libraries were also performed through a Chi square analysis on the averages of the normal and tumor SAGE tag counts. Confidence interval levels of 90%, 95% and 99% were also used for tag filtering and generated 4,030 and 762 tags, respectively. Hierarchical clustering and Venn diagrams were performed on filtered libraries using GeneSpring software release 5.0.2 build number 954 (Silicon Genetics, Redwood City, Calif.). Pearson correlation was for similarity measurement and the minimum distance was set to 0.001.

Gene expression in the aEPC was compared with gene expression from human tumor endothelial cells and with gene expression in HMVEC using SAGE analysis (33-34) (FIG. 7). Human tumor endothelial cells were derived from 3 breast tumors, 3 brain tumors and 1 colon tumor. The seven tumor endothelial cell SAGE libraries were compared with the corresponding normal tissue endothelial cell SAGE libraries and the gene expressed at significantly higher levels in the tumor endothelial cells were determined by Chi square analysis. The genes expressed in the aEPC and HMVEC as determined by SAGE analysis were compared with the genes expressed at three levels of stringency in the pooled tumor endothelial cell libraries. At each level of stringency, ≧99%, ≧95% and ≧90%, there were a larger number of expressed genes in common between the aEPC and the tumor endothelial cells than between the HMVEC and the tumor cells. Thus, at the gene expression level, there was greater similarity between aEPC and tumor endothelial cells than between HMVEC and tumor endothelial cells.

### Example 10

### Acetylated-LDL Uptake

aEPC's were tested for uptake of acetylated LDL and binding to UEA-1 lectin, traits that are common for mature endothelial cells such as HUVEC and HMVEC. The cells were incubated for 4 hours at 37° C. with 10 μg/ml Dil-Ac-LDL (Biomedical Technologies, Stoughton, Mass.) or with FITC labeled UEA-1 lectin (Sigma) for 1 hour at 37° C. in serum free media. All cells were washed twice with PBS after incubation. While HMVEC and HUVEC demonstrated robust uptake of AcLDL and binding of UEA-1 lectin, the aEPC did not take up AcLDL and weakly bound UEA-1 lectin (results not shown). The differences between cell-surface markers expressed by the aEPC and the HUVEC and HMVEC suggest that the aEPC population derived in cell culture does not express all of the characteristic markers associated with fully mature endothelial cells. Thus, aEPC may be regarded as representing an intermediary cell type between the AC133+/CD34+ progenitor cell and the mature well-differentiated endothelial cell.

### Example 11

### Functional Characterization of the Adherent Endothelial Precursor Cells

The properties of endothelial precursor cells were characterized in comparison to normal, adult endothelial cells that are commonly utilized in the angiogenesis field: HMVECs and HUVECs. aEPCs were evaluated for their ability to form tubules, migrate and invade, relevant activities not only involved with neovascularization in a wound healing setting but also with tumor development and metastasis. These assays are routinely employed to identify and evaluate both pro-angiogenic and anti-angiogenic agents that may be potentially effective in a clinical setting.

Matrigel™ matrix contains a mixture of basement membrane proteins and growth factors that induces tubule formation from single cell suspensions. The ability of cells to form tubes is a hallmark of endothelial cells that is required in order to develop vasculature with circulating blood flow. aEPCs were seeded on Matrigel™ matrix to determine if tube formation would occur as would HMVECs and HUVECs. The starting population of CD34⁺/AC133⁺ bone marrow cells were also included for comparison.

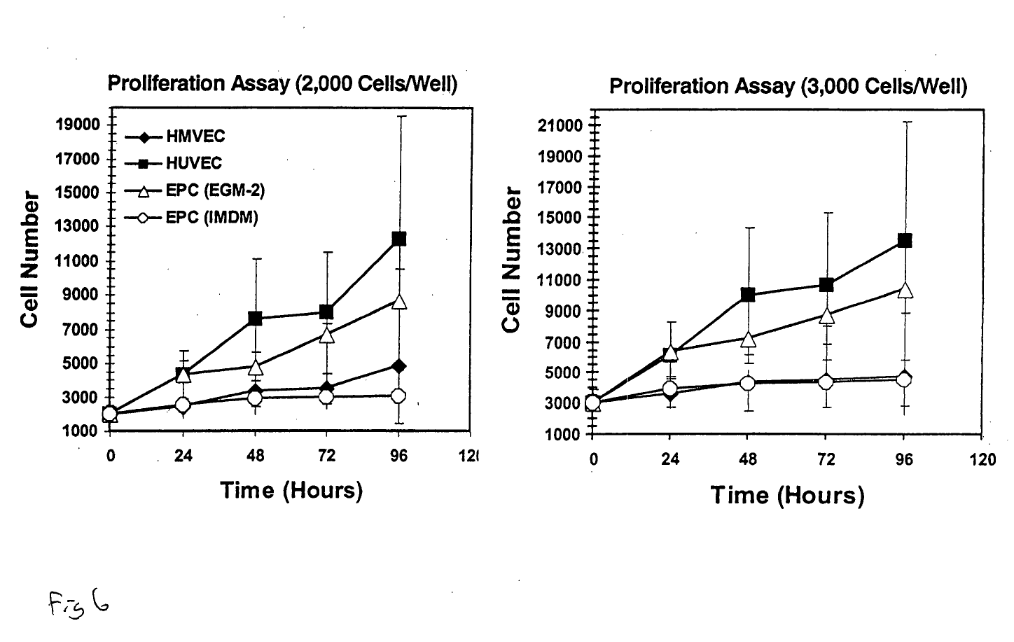

Matrigel (BD Biosciences) was added to the wells of a 48 well plate in a volume of 150 μl and allowed to solidify at 37° C. for 30 minutes. After the Matrigel solidified, aEPC, HUVEC or HMVEC (2-2.5×10³ cells) were added in 300 μl of media: IMDM with 2% FBS for aEPC and AC133+/CD34+ bone marrow progenitor cells, EGM-2 for HMVEC and HUVEC. The cells were incubated at 37° C. with humidified 95% air/5% CO₂ for 24 hours (35). The tube networks were stained with calcein and quantified by image analysis using Scion image as fluorescent pixel area. As shown in FIG. 8, it is evident that aEPCs can form tubes in a manner similar to HMVECs and HUVECs. in a 24 hour period. The CD34⁺/AC133⁺ progenitor cells were unable to form tubes on Matrigel™ matrix indicating that upon stimulation with pro-angiogenic factors the bone marrow cells selected had differentiated into a new phenotype of cells that more closely resemble HMVECs and HUVECs.

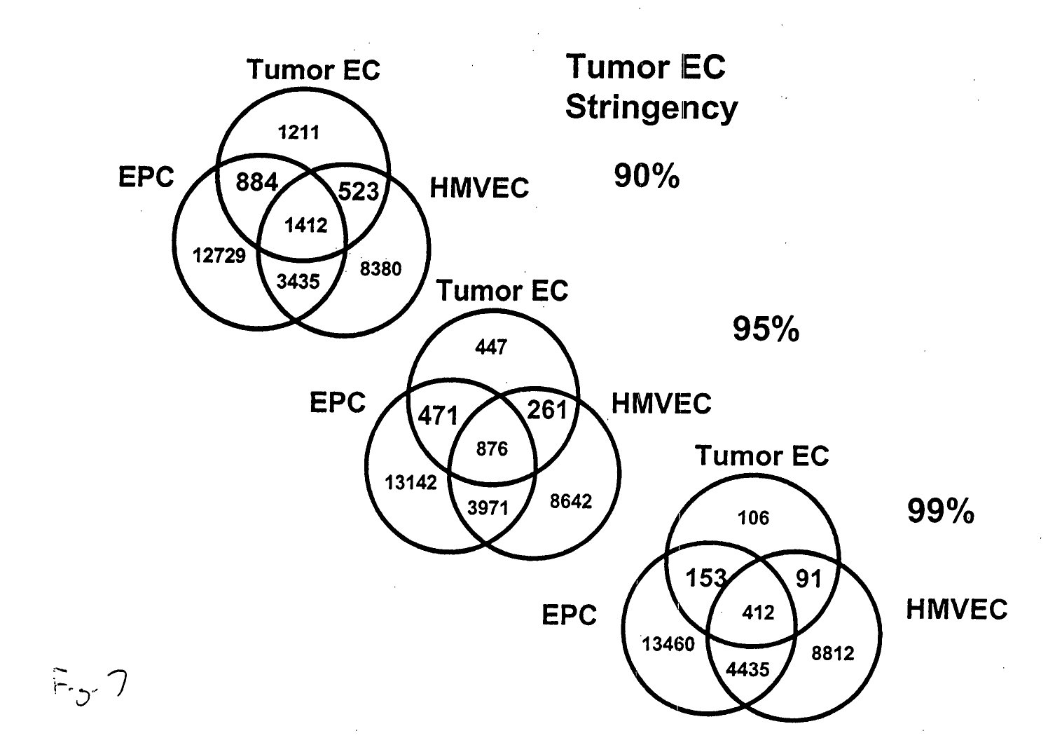

CD34+/VEGFR2+ cells from peripheral blood have been reported to migrate in response to VEGF and FGF leading to further differentiation and maturation of a subset of those progenitor cells that were expressing AC133. (Peichev et al 2000). In a subsequent manner, the adherent EPCs generated from our CD34+/AC133+ bone marrow cells stimulated with VEGF and FGF were investigated in a transmigration assay designed to evaluate their chemotactic capacity. In this assay cells in serum-free media are seeded into an insert that had been placed in a well containing a chemo-attractant that in this case was FBS at increasing concentrations. Because the insert is comprised of a light impermeable PET membrane, only the calcein labeled, migrated cells on the underside of the insert are detected. FIG. 9 (left) depicts the time course of aEPC migration over 48 hours in the presence of 0-20% serum. aEPCs begin to migrate within four hours in a serum-dependent manner and continue to migrate up to 48 hours at which point there is presumably no longer a gradient between the upper and lower chambers of the assay. Because these aEPCs will not proliferate significantly in a near serum-free environment in such a brief amount of time, the increase in fluorescence over time indicates a continued path of migration by the aEPCs. Furthermore, the aEPCs will migrate even in the total absence of serum as is shown in FIG. 9 (left).

When 0.5% FBS was used as the chemo-attractant at 24 and 48 hours, the number of cells migrating was very similar when aEPC were compared with HUVEC and HMVEC (FIG. 9B). After the aEPC population of cells was established by passing twice in the presence of the endothelial growth factors, VEGF¹⁶⁵, rhbFGF and heparin, the aEPC were maintained without addition of growth factors to the media. To determine whether continuous stimulation with VEGF⁶⁵, rhbFGF and heparin, would affect the aEPC behavior in culture, some aEPC were maintained in growth factor rich media for an additional three cell passages. As can be seen in FIG. 9B, there was no difference in ability to migrate between aEPC maintained without growth factors and those maintained in growth factor rich media.

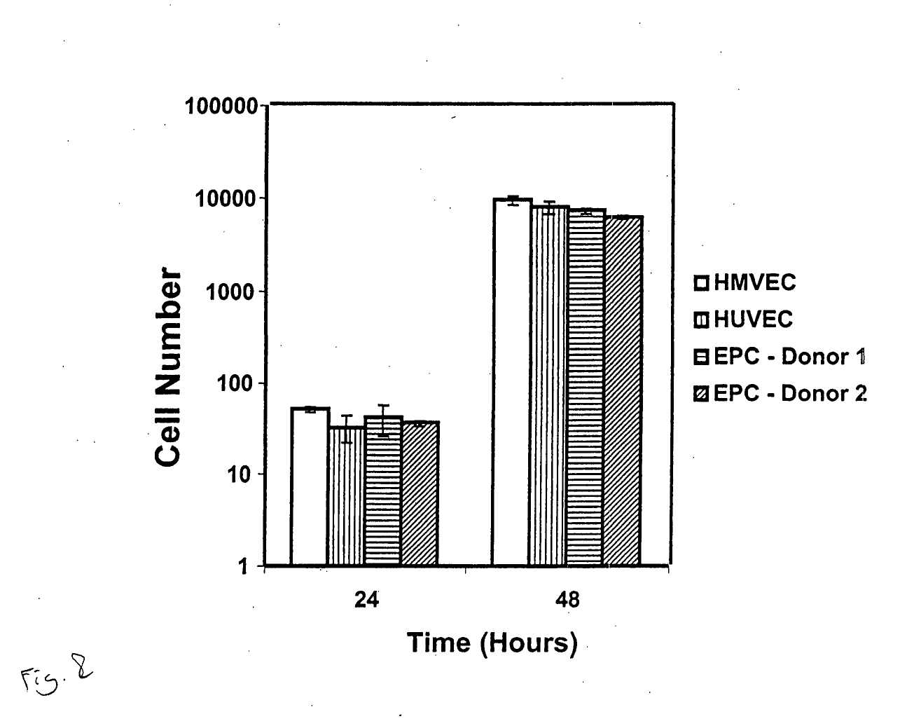

Invasion through Matrigel is another important property recognized as a characteristic of endothelial cells. The invasion assay utilized the same insert and well apparatus as the migration assay except that a layer of Matrigel coating the porous membrane through which the cells invade before they can migrate through the pores of the membrane. The invasion by aEPC, HUVEC and HMVEC was examined at 24 and 48 hours with 0.5% FBS as the chemo-attractant (FIG. 10). The aEPC performed as well as the mature endothelial cells, HUVEC and HMVEC, in the cell culture Matrigel invasion assay. In addition, AC133+/CD34+ bone marrow progenitor cells from a second donor were differentiated in aEPC. The EPC generated from both individual donors performed equally well in the cell culture Matrigel invasion assay.

### Example 13

### Interaction of aEPCs with SKOV3 Ovarian Cancer Cells

Angiogenesis is a critical step in tumor development and the role of aEPCs in neovascularization under various pro-angiogenic environments is emerging. While existing blood vessels in close proximity to a malignant tumor can contribute to the vascularization, emerging evidence suggests aEPCs may also be involved, particularly in the earlier stages of tumor formation.

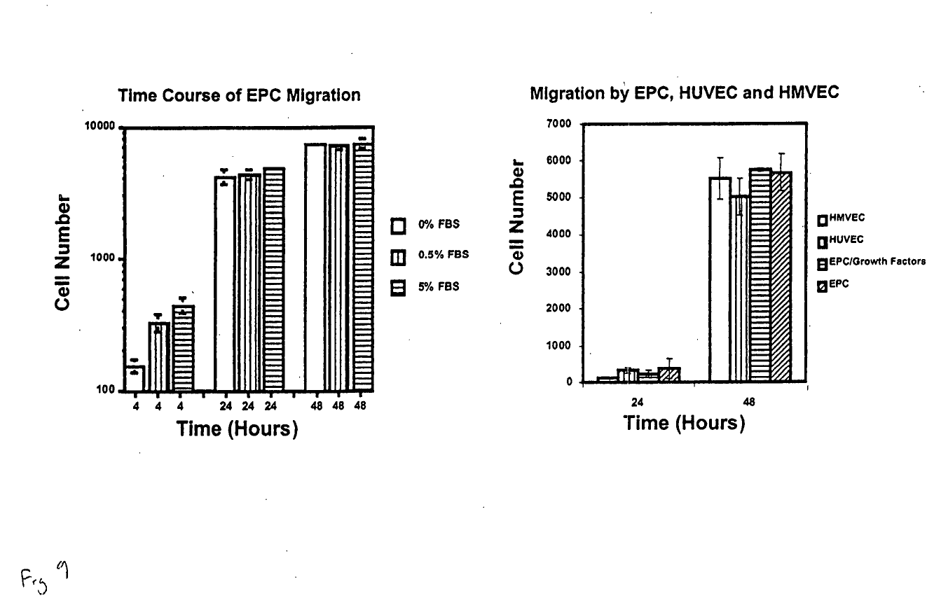

aEPCs and HVMECs were evaluated for their ability to respond to human ovarian cancer cells in a three dimensional in vitro assay. The cancer cells are clustered within a collagen plug surrounded by Matrigel™ matrix. Fluorescently-labeled aEPCs or HMVECs are added as a single cell suspension and their mobility is monitored for 48 hours. As shown in FIG. 11, aEPCs but not HMVECs were able to invade SKOV3 human ovarian cancer cell clusters suggesting that aEPCs can play a role in tumor neovascularization at even the earliest of stages. The aEPCs invaded towards the center of the clusters, an area that may becoming hypoxic as the cancer cells continue to thrive and proliferate.

While both cell types displayed invasive properties in the transwell invasion assay, only the aEPCs responded to signals released by the ovarian cancer cells. Recruitment of endothelial cells for neovascularization of a growing tumor may initially target the younger, less mature EPCs that have greater potential for differentiation and may home more rapidly to the site of angiogenesis. Subsequent growth and development of vessels could then rely upon existing vasculature for support. Identifying which cancer cell types are more likely to produce factors that will recruit aEPCs may assist in the diagnosis of more aggressive tumors.

### Example 14

### Matrigel Angiogenesis Assay

The ability to form tubes or networks in Matrigel is a hallmark of endothelial cell behavior that models the formation of new vessels or vasculature in vivo. For the tube/network formation assay, AC133+/CD34+ bone marrow progenitor cells, EPC, HUVEC and HMVEC were plated onto a layer of Matrigel and allowed to incubate for 24 hours (FIG. 10). The more undifferentiated AC133+/CD34+ bone marrow progenitor cells did not form tubes or networks on the Matrigel. However, the EPC formed tubes/networks that appear quite similar to those formed by HUVEC and HMVEC. Thus, differentiation of the CD34⁺/AC133⁺ bone marrow progenitor cells toward the endothelial cell phenotype as represented by EPC allows the cells to form tubes/networks on Matrigel indicating that upon exposure to pro-angiogenic factors cells derived from bone marrow can develop several properties similar to mature endothelial cells like HUVEC and HMVEC.

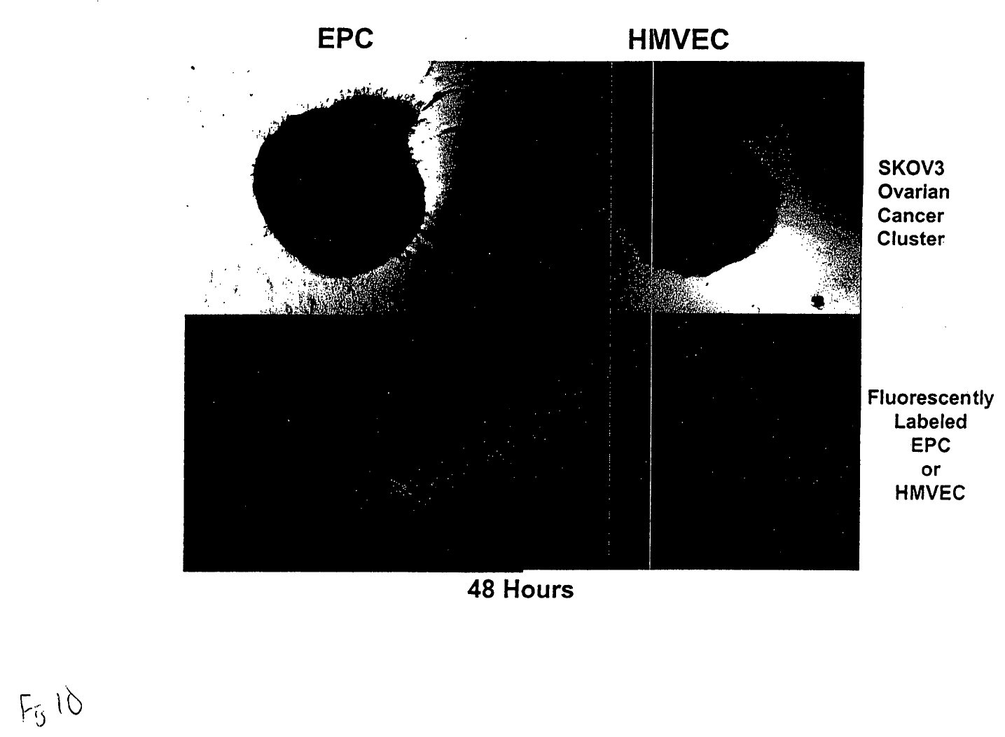

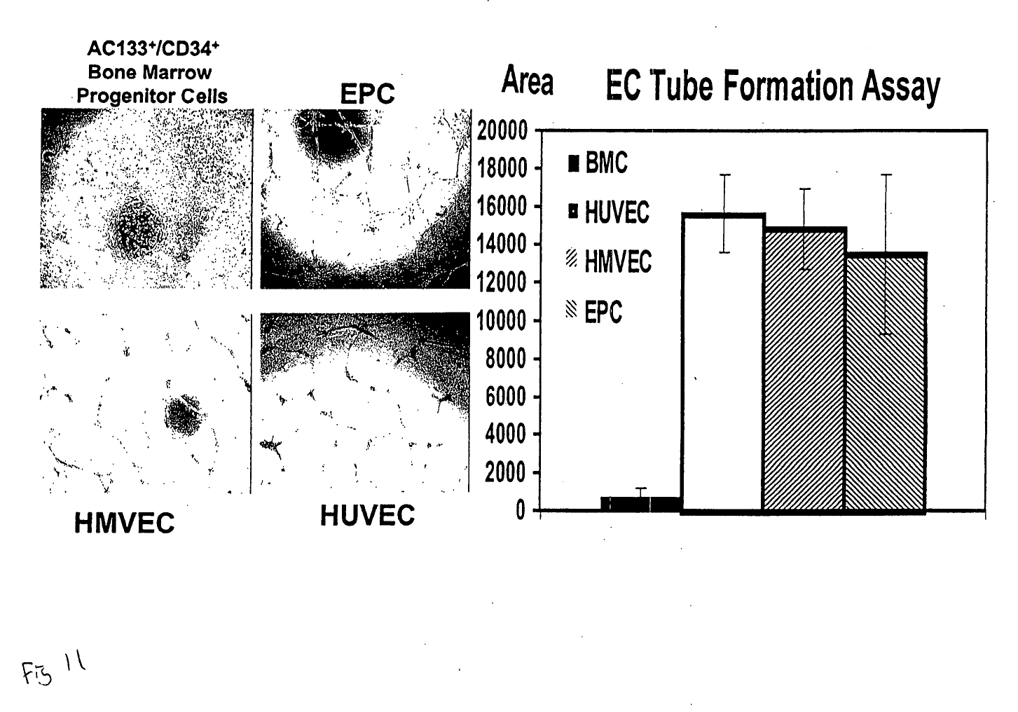

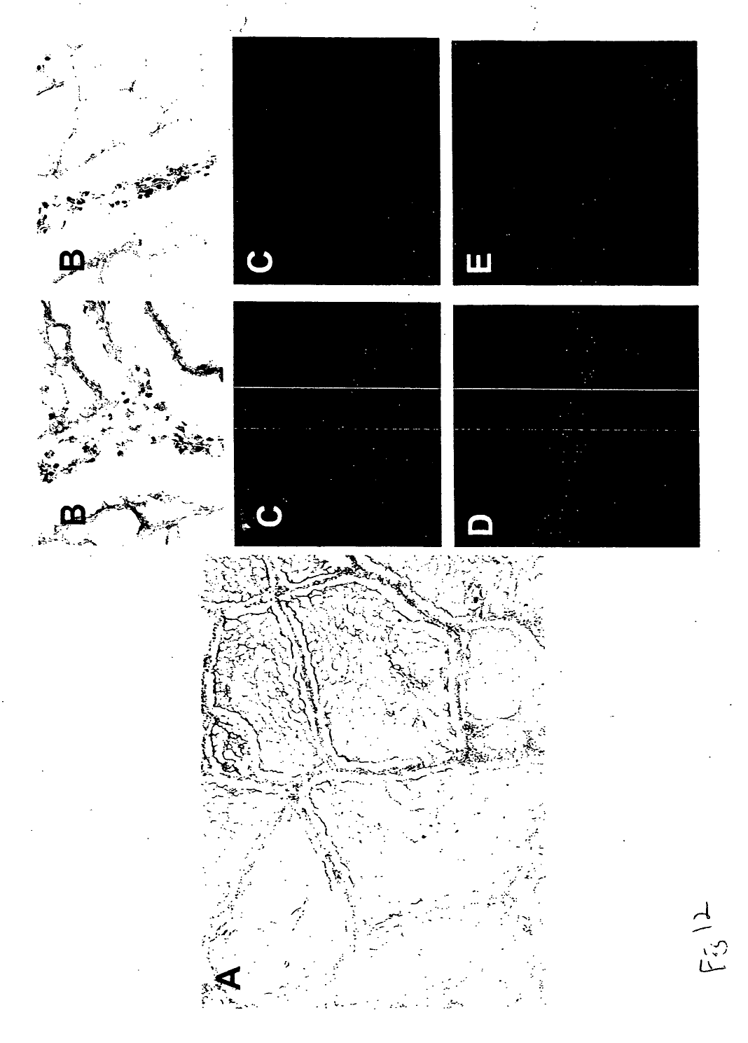

To develop a convenient in vivo model for testing potential antiangiogenic agents against human vascular targets expressed on EPC, EPC (5×10⁵ cells) labeled with a tracer amount of the fluorescent nuclear-stain DAPI were mixed into Matrigel (500 μl) and injected subcutaneously into nude mice. After 7 days, the cell-laden Matrigel plugs were collected and snap frozen. Sections from the plugs were evaluated for tube/network formation and retention of the EPC (FIGS. 12A-E). The tubes/network formed throughout the plugs and apparent degradation of the Matrigel support was visualized by staining with hematoxylin and eosin (FIGS. 12A and 12B). Fluorescence microscopy allowed visualization of the nuclei of DAPI-labeled EPC in the tubes/network (FIG. 12C). FIG. 12D shows staining of the EPC for CD31 and FIG. 12E shows staining for von Willebrand's factor by fluorescent immunohistochemistry.

Because Matrigel™ matrix can induce tube formation from murine host endothelial cells alone, one may presume that the vasculature that has formed is a chimera of both human and mouse cells. Visualization of the DAPI-labeled aEPCs, revealed that there were some regions of the vasculature that were not comprised of aEPCs but rather consisted of murine endothelial cells. The anastomoses of the aEPCs and host endothelial cells has generated a basic model of human vasculature in a murine host without the need of surgical methods or artificial, solid supports. Pre-clinical models comprised at least in part of endothelial cells of human origin are valuable in evaluating the efficacy of potential anti-angiogenic therapeutics.

The aEPC were obtained from bone marrow cells expressing CD34 and AC133, however, the full potential of this subpopulation of progenitor cells remains to be elucidated. While expression of AC133 protein appears to be limited to bone marrow and some leukemias from immunohistochemical staining, the message for AC133 is present in other tissues including kidney and pancreas (37). It is possible that under specific stimulatory conditions that AC133+ progenitor cells can differentiate into various cell types. A second isoform of AC133 expressed in human stem cells other than hematopoietic tissue has been identified (38).

The aEPC examined in these studies are likely intermediary between early progenitor cells and fully mature endothelial cells. Like HUVEC and HMVEC, EPC have the capacity to migrate, invade through Matrigel and form tubes/networks on a Matrigel-coated substrate. The in vivo environment cannot be wholly mimicked in culture and all of the components that contribute to the maturation and maintenance of endothelial cells have yet to be fully characterized. However, there was a clear difference in the behavior of aEPC and HMVEC in the co-culture assay where human SKOV3 ovarian cancer cells provided the stimulus for vasculogenesis/neoangiogenesis. In that assay, the aEPC were able to invade into the tight cluster of malignant cells while the HMVEC did not have the capacity for invasion. SAGE analysis for gene expression allowed us to compare aEPC and HMVEC to gene expression in tumor endothelial cells isolated from clinical surgical samples of breast, colon and brain cancer. The data show that aEPC are more similar in expressed genes to tumor endothelial cell than are HMVEC. Loading human aEPC into Matrigel and the injection of the Matrigel as a subcutaneous plug into murine hosts resulted in formation of a network/vasculature that was likely a mosaic of human and mouse cells after 7 days. Upon visualization of the DAPI-labeled aEPC, it was evident from the presence of unlabeled cells that some regions of the vasculature that were not comprised of aEPC but rather consisted of murine endothelial cells. Another possibility is that host macrophages could enter the Matrigel and engulf the human aEPC; however, the number of pyknotic cells in the Matrigel plugs was very low (0.1%) and macrophage-like cells were seen. The anastomses of the aEPC and host endothelial cells has generated a basic model of human vasculature in a murine host. Preclinical models comprised at least in part of endothelial cells of human origin are valuable in evaluating the efficacy of potential anti-angiogenic therapeutics. This model is, however, simpler to execute than bone marrow transplant or skin xenograft models (43-46).

### REFERENCES

1. Van der Kolk S. IN: Blood Supply of Tumors, vol. 2 (Montagna W and Ellis R, eds.) pp. 123-149, 1826.
2. Jones T. Guy's Hospital Reports, 2^{nd} Ser 7: 1-94, 1850.
3. Paget S. Lancet March 23: 571-573, 1989.
4. Algire G and Chalkey H. J Natl Cancer Inst 6: 73-95, 1945.
5. Folkman M J, Merler E, Abernathy C, Williams G. Isolation of a Tumor Factor Responsible for Angiogenesis. J Exp Med 133: 275-288, 1971.
6. Folkman M J. Adv Cancer Res 19: 331-358, 1974.
7. Folkman M J, Cotran R. Int Rev Exp Pathol 16: 207-248, 1976.
8. Folkman M J. New Engl J Med 285: 1182-1186, 1971.
9. Auerbach W, Auerbach R. Angiogenesis inhibition: a review. Pharmacol Therap 63: 265-311, 1994.
10. Modzelewski R A, Davies P, Watkins S C, Auerbach R. Chang M-J, Johnson C S. Isolation and identification of fresh tumor-derived endothelial cells from a murine RIF-1 fibrosarcoma. Cancer Res 54: 336-339, 1994.
11. Lu L, Wang S, Auerbach R. In vitro and in vivo differentiation into B Cells, T cells and myeloid cells of primitive yolk sac hematopoietic precursor cells expanded >100-fold by coculture with a clonal yolk sac endothelial cell line. Proc Natl Acad Sci USA 93: 14782-14787, 1996.
12. Asahara T, Murohara T, Sullivan A, Silver M, van der Zee R, Li T, Witzenbichler B, Schatteman G, Isner J. Isolation of putative progenitor endothelial cells for angiogenesis. Science 275: 964-967, 1997.
13. Asahara T, Masuda H, Takahashi T, Kalka C, Pastore C, Silver M, Kearne M, Magner M, Isner J. Bone marrow origin of endothelial progenitor cells responsible for postnatal vasculogenesis in physiological and pathological neovascularization. Circ Res 85: 221-228, 1999a.
14. Asahara T, Takahashi T, Masuda H, Kalka C, Chen D, Iwaguro H, Inai Y, Silver M, Isner J. VEGF contributes to postnatal neovascularization by mobilizing bone marrow-derived endothelial progenitor cells. EMBO J 18: 3964-3972, 1999b.
15. Lin Y, Weisdorf D J, Solovey A, Hebbel R P. Origins of circulating endothelial cells and endothelial outgrowth from blood. J Clin Invest 105: 71-77, 2000.
16. Gehling U M, Ergun S, Schumacher U, Wagener C, Pantel K, Otte M, Schuch G, Schafhausen P, Mende T, Kilic N, Kluge K, Schafer B, Hossfeld D, Fiedler W. In vitro differentiation of endothelial cells from AC133-positive progentior cells. Blood 95: 3106-3112, 2000.
17. Kaufman D, Hanson E, Lewis R, Auerbach R, Thomson J. Hematopoietic colony-forming cells derived from human embryonic stem cells. Proc Natl Acad Sci USA 98: 10716-10721, 2001.
18. Auerbach R, Akhtar N, Lewis L, Shinners B L. Angiogenesis assays: problems and pitfalls. Cancer Metas Rev 19: 167-172, 2000.
19. Rafii S. Circulating endothelial precursors: mystery, reality, and promise. J Clinic Invest 105: 17-19, 2000.
20. Peichev M, Naiyer A, Pereira D, Zhu Z, Lane W, Williams M, Oz M, Hicklin D, Witte L, Moore M, Rafii S. Expression of VEGFR-2 and AC133 by circulating human CD34+ cells identifies a population of functional endothelial precursors. Blood 95: 952-958, 2000.
21. Gill M, Dias S, Hattori K, Rivera, M, Hicklin D, Witte L, Girardi L, Yurt R, Himiel H, Rafii S. Vascular trauma induces rapid but transient mobilization of VEGFR2+AC133+ endothelial precursor cells. Circ Res 88: 167-174, 2001.
22. Lyden D, Hattori K, Dias S, Costa C, Blaikie P, Butros L, Chadburn A, Heissig B, Marks W, Witte L, Wu Y, Hicklin D, Zhu Z, Hackett N, Crystal R, Moore M, Hajjar K, Manova K, Benezra R, Rafii S. Impaired recruitment of bone-marrow-derived endothelial and hematopoietic precursor cells blocks tumor angiogenesis and growth. Nature Medicine 7: 1194-1201, 2001.
23. De Bont E, Guikema J, Scherpeh F, Meeuwsen T, Kamps W, Vellenga E, Bos N. Mobilized human CD34+ hematopoietic stem cells enhance tumor growth in a nonobese diabetic/severe combined immunodeficient mouse. Cancer Res 61: 7654-7659, 2001.
24. Luttun A, Carmeliet G, Carmeliet P. Vascular progenitors: from biology to treatment. Trends Cardiovasc Med 12: 88-96, 2002.
25. Reyes M, Dudek A, Jahagirdar B, Koodie L, Marker P, Vefaillie C. Origin of Endothelial progenitors in human postnatal bone marrow. J Clin Invest 109: 337-346, 2002.
26. Hiessig B, Hattori K, Dias S, Friedrich M, Ferris B, Hackett N, Crystal R, Besmer P, Lyden D, Moore M, Werb Z, Rafii S. Recruitment of stem and progenitor cells from the bone marrow niche requires MMP-9 mediated release of Kit-Ligand. Cell 109: 625-637, 2002.
27. Shirakawa K, Furuhata S, Watanabe I, Hayase H, Shimizu A, Ikarashi Y, Yoshida T, Terada M, Hashimoto D, Wakasugi H. Induction of vasculogenesis in breast cancer models. Brit J Cancer 87: 1454-1461, 2002.
28. Capillo M, Mancuso P, Gobbi A, Monestiroli S, Pruneri G, Dell'Agnla C, Martinelli G, Shultz L, Bertolini F. Continuous infusion of endostatin inhibits differentiation, mobilization, and clonogenic potential of endothelial cell progenitors. Clin Cancer Res 9: 377-382, 2003.
29. Miraglia S, Godfrey W, Yin A, Atkins K, Warnke R, Holden J, Bray R, Waller E, Buck D. A novel five-transmembrane hematopoietic stem cell antigen: isolation, characterization, and molecular cloning. Blood 90: 5013-5021, 1997.
30. Pelletier L, Regnard J, Fellmann D, Charbord P. An in vitro model for the study of human bone marrow angiogenesis: role of hematopoietic cytokines. Lab Invest. 80: 501-511, 2000.
31. Quirici N, Soligo D, Caneva L, Servida F, Bossolasco P, Deliliers G. Differntiation and expansion of endothelial cells from human bone marrow CD133+ cells. Brit J Haematol 115: 186-194, 2001.
32. Gehling U, Ergun S, Schumacher U, Wagener C, Pantel K, Otte M, Schuch G, Schafhausen P, Mende T, Kilic N, Kluge K, Schafer B, Hossfel D, Fiedler W. In vitro differentiation of endothelial cells from AC133-positive progenitor cells. Blood 95: 3106-3112, 2000.
33. Bastaki M, Nelli E E, Dell'Era P, Rusnati M, Molinari-Tosatti M P, Parolini S, Auerbach R, Ruco L P, Possati L, Presta M. Basic fibroblast growth factor-induced angiogenic phenotype in mouse endothelium: a study of aortic and microvascular endothelial cell lines. Aterioscler Thromb Vasc Biol 17: 454-464, 1997.
34. Gerwins P, Skildenberg E, Claesson-Welsh, L. Function of fibroblast growth factors and vascular endothelial growth factors and their receptors in angiogenesis. Crit Rev Oncol Hematol 34: 185-194, 2000.
35. Muller A, Hermanns M, Skrzynski C, Nesslinger M, Muller K, Kirkpatrick C. Expression of the endothelial markers PECAM-1, vWF, and CD34 in vivo and in vitro. Exp Molec Pathol 72: 221-229, 2002.
36. Compagni A, Wilgenbus P, Impagnatiello M, Cotton M, Christofori G. Fibroblast growth factors are required for efficient tumor angiogenesis. Cancer Res 60: 7163-7169, 2001.
37. Shirakawa K, Shibuya M, Heike Y, Takashima S, Watanabe I, Konishi F, Kasumi F, Goldman C, Thomas K, Bett A, Terada M, Wakasugi H. Tumor-infiltrating endothelial cells and endothelial precursor cells in inflammatory breast cancer. Int J Cancer 99: 344-351, 2002.
38. Murohara T, Ikeda H, Duan J, Shintani S, Sasaki K, Eguchi H, Onitsuka I, Matsui K, Imaizumi T. Transplanted cord blood-derived endothelial precursor cells augment postnatal neovascularization. J Clin Invest. 105: 1527-1536, 2000.
39. Vacca A, Ribatti D, Roccaro A, Frigeri A, Dammacco F. Bone marrow angiogenesis in patients with active multiple myeloma. Semin Oncol 28: 543-550, 2001.
40. Bian X, Du L, Shi J, Cheng Y, Liu F. Correlation of bFGF, FGFR-1 and VEGF expression with vascularity and malignancy of human astrocytomas. Anal Quant Cytol Histol 22: 267-274, 2000.
41. Cross M. and Claesson-Welsh, L. (2001) FGF and VEGF Function in Angiogenesis: Signaling Pathways, Biological Responses and Therapeutic Inhibition. Trends in Pharmacological Sciences, Vol. 22(4), 201-207.
42. Folkman, M J. (1971b) Tumor Angiogenesis: Therapeutic Implications. New England Journal of Medicine, Vol. 285, 1182-1186.
43. Yu Y., Flint A., Dvorin E., and Bischoff J. (2002) AC133-2, a Novel Isoform of Human AC133 Stem Cell Antigen. Journal of Biological Chemistry, Vol. 277(23), 20711-20716.
44. Morikawa, S., Baluk, P., Kaidoh, T., Haskell, A., Jain, R. K., and McDonald, D. M. Abnormalities in pericytes on blood vessels and endothelial sprouts in tumors. Amer. J. Pathol., 160: 985-1000, 2002.
45. Thurston, G., Suri, C., Smith, K., McClain, J., Sato, T. N., Yancopoulos, G. D., and McDonald, D. M. Leakage-resistant blood vessels in mice transgenically overexpressing angiopoietin-1. Science, 286: 2511-2514, 1999.
46. Brown, E. B., Campbell, R. B., Tsuzuki, Y., Xu, L., Carmeliet, P., Fukumura, D., and Jain, R. K. In vivo measurement of gene expression, angiogenesis and physiological function in tumors using multiphoton laser scanning microscopy. Nature Med., 7: 864-868, 2001.
47. O'Connell, K. A. and Edidin, M. A mouse lymphoid endothelial cell line immortalized by simian virus 40 binds lymphocytes and retains functional characteristics of normal endothelial cells. J. Immunol., 144: 521-525, 1990.
48. O'Connell, K., Landman, G., Farmer, E., and Edidin, M. Endothelial cells transformed by SV40 T antigen cause Kaposi's sarcomalike tumors in nude mice. Amer. J. Pathol., 139: 743-749, 1991.
49. O'Connell, K. A. and Rudman, A. A. Cloned spindle and epitheliodid cells from murine Kaposi's sarcoma-like tumors are of endothelial origin. J. Invest. Dermatol., 100: 742-745, 1993.
50. St. Croix, B., Rago, C., Velculescu, V., Traverso, G., Romans, K. E., Montgomery, E., Lal, A., Riggins, G. J., Lengauer, C., Vogelstein, B., and Kinzler, K. W. Genes expressed in human tumor endothelium. Science, 289: 1197-1202, 2000.
51. Carson-Walter, E. B., Watkins, D. N., Nanda, A., Vogelstein, B., Kinzler, K. W., and St. Croix, B. Cell surface tumor endothelial markers are conserved in mice and humans. Cancer Res., 61: 6649-6655, 2001.
52. Walter-Yohrling J, Pratt B M, Ledbetter S, Teicher B A. Myofibroblasts enable invasion of endothelial cells into 3-dimensional tumor cell clusters: a novel in vitro tumor model. Cancer Chemother Pharmacol, in press, 2003.

1. An isolated population of adherent human EPCs made by the process of:
stimulating human bone marrow cells expressing endothelial lineage markers AC133 and CD34 with VEGF, bFGF, and heparin.

2. An isolated population of adherent human EPCs which are capable of invading human ovarian cancer cells clusters in a three dimensional in vitro assay.

3. The population of claim 1 or claim 2 which does not express endothelial lineage markers AC133 or CD34.

4. The population of claim 1 or claim 2 which has been separated from non-adherent cells.

5. The population of claim 1 or claim 2 which does not take up acetylated LDL (low density lipoprotein).

6. A method of evaluating test agents as pro-angiogenic or anti-angiogenic factors, comprising:
contacting an isolated population of adherent human EPCs according to claim 1 or claim 2 with a test agent;
evaluating tubule formation, migration, or invasion by the isolated population contacted with the test agent relative to an isolated population not contacted with a test agent; and
identifying the test agent as having potential use as a pro-angiogenic factor if it increases tubule formation, migration, or invasion, and identifying the test agent as having potential use as an anti-angiogenic factor it decreases tubule formation, migration, or invasion.

7. A method of stimulating AC133⁺/CD34⁺ human bone marrow cells, comprising:
culturing the cells on a collagen-coated surface in the presence of FBS, VEGF, and heparin.

8. The method of claim 7 further comprising the step of separating adherent cells from non-adherent cells.

9. A method of stimulating AC133⁺/CD34⁺ human bone marrow cells, comprising:
culturing the cells on a fibronectin-coated surface in the presence of FBS, VEGF, FGF, and heparin.

10. The method of claim 9 further comprising the step of separating adherent cells from non-adherent cells.

11. A model system for human vasculature, comprising:
a nude mouse which has been co-injected with reconstituted basement membrane matrix and the isolated population of adherent human EPCs of claim 1 or claim 2.

12. A model system for human vasculature comprising a sample of Matrigel and aEPCs which has been removed from the nude mouse of claim 11.

13. The model system of claim 11 wherein the EPCs are labeled prior to injection into the nude mouse.

14. The model system of claim 12 wherein the aEPCs are labeled prior to injection into the nude mouse.

15. A method of identifying a mouse cell line useful as a model for tumor endothelial cells, comprising:
determining expression of two or more murine tumor endothelial markers in one or more mouse cell lines;
selecting a mouse cell line which expresses at least two of said markers more than it expresses 18S RNA.

16. A method of evaluating test agents as pro-angiogenic or anti-angiogenic factors, comprising:
contacting a mouse cell line selected by the method of claim 15 with a test agent;
evaluating tubule formation, migration, or invasion by the mouse cell line contacted with the test agent relative to the mouse cell line not contacted with a test agent; and
identifying the test agent as having potential use as a pro-angiogenic factor if it increases tubule formation, migration, or invasion, and identifying the test agent as having potential use as an anti-angiogenic factor it decreases tubule formation, migration, or invasion.


SEQUENCE LISTING (16 sequences)

SEQ ID NO: 1 - DNA, 20 bp, Homo sapiens
gaagacccttattacacgga

SEQ ID NO: 2 - DNA, 17 bp, Homo sapiens
gctgaatggccgtttct

SEQ ID NO: 3 - DNA, 21 bp, Homo sapiens
cagcaagcgggagcccgtggt

SEQ ID NO: 4 - DNA, 21 bp, Homo sapiens
ggtgctctgggtgctcccgat

SEQ ID NO: 5 - DNA, 21 bp, Homo sapiens
gcagaggactggccatttgga

SEQ ID NO: 6 - DNA, 26 bp, Homo sapiens
gcaacagctcgatatctgtcaatact

SEQ ID NO: 7 - DNA, 20 bp, Homo sapiens
tggagatagtgagccttgga

SEQ ID NO: 8 - DNA, 23 bp, Homo sapiens
cagtttcgaggctgctccatgcg

SEQ ID NO: 9 - DNA, 23 bp, Homo sapiens
gagattgttagcttaggaggcac

SEQ ID NO: 10 - DNA, 27 bp, Homo sapiens
gtctcattagatcatacacctcatcat

SEQ ID NO: 11 - DNA, 22 bp, Homo sapiens
gacctgttccagcgagggtcta

SEQ ID NO: 12 - DNA, 27 bp, Homo sapiens
cttccatcctcatagcaattaaggtgg

SEQ ID NO: 13 - DNA, 21 bp, Homo sapiens
ctgggagcctgcacgaagcaa

SEQ ID NO: 14 - DNA, 23 bp, Homo sapiens
gtcacgtttgctcttgaggtagt

SEQ ID NO: 15 - DNA, 25 bp, Homo sapiens
gcagacagaaatacgtttgagttgg

SEQ ID NO: 16 - DNA, 19 bp, Homo sapiens
agtgattgccccatgtgga